(19) United States
(12) Patent Application Publication
(10) Pub. No.: US 2025/0292912 A1
(43) Pub. Date: Sep. 18, 2025

(54) SYSTEMS, DEVICES AND METHODS FOR HEALTH MONITORING AND IDENTIFICATION OF USERS

(71) Applicant: Casana Care, Inc., Rochester, NY (US)

(72) Inventors: Nicholas Joseph CONN, Fairport, NY (US); Kyle HUGGINS, Rochester, NY (US); Andrea KRAAY, San Diego, CA (US); David A. BORKHOLDER, Canandaigua, NY (US)

(21) Appl. No.: 19/228,396

(22) Filed: Jun. 4, 2025

Related U.S. Application Data
(63) Continuation of application No. PCT/US2024/014878, filed on Feb. 7, 2024.
(60) Provisional application No. 63/443,903, filed on Feb. 7, 2023.

(51) Int. Cl. G16H 50/30; A47K 13/24

(52) U.S. Cl. CPC G16H 50/30; A47K 13/24

(57) ABSTRACT

Systems, devices, and methods are disclosed herein for measuring sensor data of a user seated on a toilet and detecting/identifying the user based on the measured sensor data using statistical models and/or machine learning. In some embodiments, the systems described herein can include multiple sensors including force sensor(s), ECG sensor(s), PPG sensor(s), and other sensor(s), disposed in and/or integrated to a toilet. The sensors can measure and/or record sensor data including loads and forces, BCG-data, ECG-data, PPG data, bioimpedance, or the like, and determine, based on the sensor data, one or more physiological parameters and/or characteristics about the user. The systems can be further configured to associate the measured sensor data and the biological conditions and/or characteristics with a user identifier such that the user can be identified by comparing the user identifier with sensor data measured when a subject (e.g., an unknown subject) is seated on the toilet.

### CROSS-REFERENCE TO RELATED APPLICATIONS

This application is a continuation of International Patent Application No. PCT/US2024/014878, entitled “Systems, Devices and Methods for Health Monitoring and Identification of Users”, filed Feb. 7, 2024, which claims priority to and the benefit of U.S. Provisional Patent Application No. 63/443,903, entitled “Systems and Methods for Health Monitoring and Identification of Users,” filed Feb. 7, 2023, the disclosures of which are incorporated by reference herein in their entireties.

### TECHNICAL FIELD

The embodiments described herein relate generally to health monitoring systems, and more particularly to systems and methods for monitoring user features or characteristics and performing user identification.

### BACKGROUND

Patient health monitoring is an important tool in tracking physiological conditions of patients and to provide early warnings or guidance to individuals and healthcare providers in cases of patient health deterioration. Oftentimes, patient monitoring is obtrusive and requires individuals to actively wear certain devices or change their routine to be able to measure certain vital signs or characteristics of the patient. Unobtrusive systems and/or devices for monitoring individuals are limited. Furthermore, in multiple instances these systems and/or devices are used and/or shared between multiple subjects, individuals, and/or users as for example, in a group and/or household. Consequently, each subject, individual and/or user included in the group and/or household may be required to routinely identify him or herself prior to monitoring and/or measuring his/her vital signs and/or characteristics. Therefore, there exists a need to develop more accurate approaches to identify and then monitor multiple subjects, individuals and/or users using unobtrusive systems.

### SUMMARY

Systems, devices, and methods are described herein for identifying a user using measured physiological data, including measurements obtained using unobtrusive mechanisms of users near or seated on a toilet or other lavatory device.

### BRIEF DESCRIPTION OF THE DRAWINGS

FIG. 1 illustrates a schematic illustration of a sensing system for identifying a user based on one or more physical and/or physiological characteristics measured from the user, according to an embodiment.

FIG. 2A schematically depicts a network of devices for identifying a user based on one or more physical and/or physiological characteristics measured from the user, according to an embodiment.

FIG. 2B schematically depicts a network of devices for identifying multiple users based on physical and/or physiological characteristics measured from each user, according to an embodiment.

FIGS. 3A, 3B, and 3C are a top, side, and bottom view, respectively, of a toilet seat including a set of sensors for monitoring signals associated with one or more physical and/or physiological characteristics of a user (e.g., photoplethysmogram (PPG) signals, electrocardiogram (ECG) signals, loads or forces, etc.), according to an embodiment.

FIGS. 4A-4B present a flow chart of an example method of onboarding a new user of a sensing system, according to an embodiment.

FIG. 5 is a flow chart of an example method of using a sensing system to measure one or more physical and/or physiological characteristics of a user seated on a toilet and identify, based on the measured characteristics, the seated user, according to an embodiment.

FIG. 6 is a flow chart of an example method of calibrating or adapting a sensing system based on measurements of one or more physical and/or physiological characteristics of a user, according to an embodiment.

FIG. 7 is chart depicting data points of one or more users, according to embodiments.

FIG. 8 is a chart depicting changes in data points of a user over time and schematically indicating the adaptability of a sensing system to such changes, according to embodiments.

FIGS. 9A-9B are top side views of toilet seats including a set of sensors for monitoring signals associated with one or more physical and/or physiological characteristics of a user (e.g., photoplethysmogram (PPG) signals, bioimpedance electrode signals, and/or electrocardiogram signals), according to different embodiment.

### DETAILED DESCRIPTION

The embodiments described herein relate generally to health monitoring systems and devices, and more particularly to systems, devices, and methods for measuring sensor data (e.g., electrical signals such as voltages and/or electrical current, light signals, and/or forces and loads) when users are using a toilet, urinal, or other lavatory device. Such systems, devices, and methods can provide accurate measurements of sensor data including, for example, weight, a ballistocardiogram (BCG), an electrocardiogram (ECG), a photoplethysmogram (PPG), body temperature, and/or a bioimpedance of a user, which can be used to monitor certain physiological data or conditions of the user and to inform the user and/or healthcare providers of changes in such data or conditions necessitating certain therapies, treatments, lifestyle changes, etc.

Most individuals use toilets, urinals, or other defecation or urination devices on a daily basis. Accordingly, health monitoring that can be conducted while an individual is defecating and/or urinating into such devices can provide an unobtrusive way of regularly monitoring information about that individual. Measures such as a seated weight, BCG, ECG, PPG, body temperature, and/or bioimpedance of an individual can be useful for monitoring information about that individual. For example, such measures can be useful for monitoring certain conditions of the individual, such as, for example, a cardiac or vascular heath of the individual, fever, menstrual health, circadian rhythm, insomnia and sleep disturbances, and/or overall health and wellbeing.

Various sensing or monitoring systems can be used to measure one or more physical and/or physiological characteristics of a user. For example, scales can be used to measure a body weight or a body mass index (BMI) of a subject. Wearable devices can be used to measure heartbeat, oxygen level, movement, and/or other data of a subject. Such devices, however, may be obtrusive, e.g., requiring a user to incorporate the use of a device into their daily routine. However, many sensing or monitoring devices are limited to measuring one or a few different physical and/or physiological characteristics of a user, and therefore lack the comprehensive data capture for being able to effectively or accurately identify different users. In private and public settings, multiple users may also use or interact with the same sensing devices. Therefore, it can be desirable to have systems and devices that can not only measure physical and/or physiological characteristics of users but also identify and adapt with different users.

In some embodiments, systems, devices, and methods described herein can be implemented using a toilet. The toilet can include, for example, one or more sensors configured to measure physical and/or physiological characteristics of users that use or interact with the toilet. In some embodiments, systems, devices, and methods described herein can be implemented using a toilet and/or one or more other sensing devices, e.g., a scale device or a temperature measurement device. The combination of physical and/or physiological characteristics of users that can be measured by such systems and devices can provide more comprehensive assessments of an individual's health. For example, such physical and/or physiological characteristics can be used to monitor and assess conditions including, for example, an individual's respiration, body weight, body temperature, BCG, pulse wave velocity (PWV), stroke volume, cardiac output, weight of or urination or defecation and/or a weight change associated therewith, etc. Examples of sensing devices are described in U.S. Pat. No. 10,292,658, titled “Apparatus, System, and Method for Mechanical Analysis of Seated Individual,” issued May 21, 2019 (the '658 patent); U.S. Patent Application Publication No. 2022/0378373, titled “Systems, Devices, and Methods for Monitoring Loads and Forces on a Seat,” published Dec. 1, 2022 (the '373 application); U.S. Pat. No. 11,650,094, titled “Systems, Devices, and Methods for Measuring Loads and Forces of a Seated Subject Using Scale Devices,” issued May 16, 2023 (the '094 patent); U.S. Patent Application Publication No. 2022/0361754, titled “Systems, Devices, and Methods for Measuring Body Temperature of a Subject Using Characterization of Feces and/or Urine,” published Nov. 17, 2022 (the '754 application); and International Patent Application No. PCT/US2023/075553, titled “Photoplethysmography Sensing Module, Systems and Devices Thereof,” filed Sep. 29, 2023 (the '553 application). The disclosures of each of the foregoing are incorporated herein by reference in their entirety.

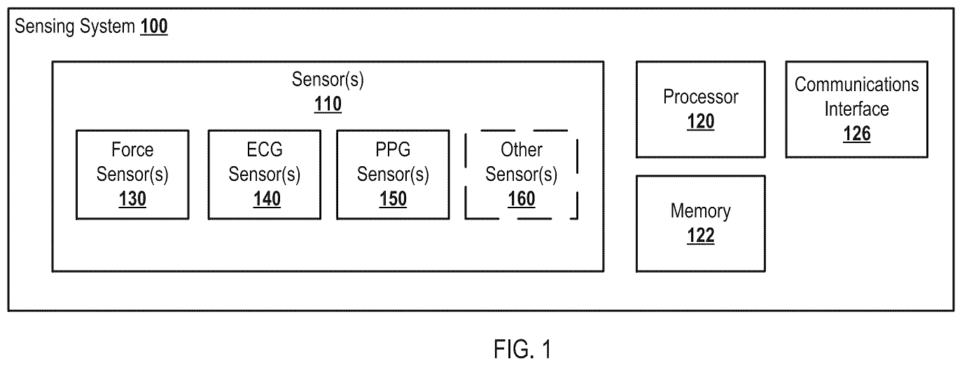

FIG. 1 shows a schematic illustration of a sensing system 100, according to embodiments. In some embodiments, the sensing system 100 can be implemented as and/or coupled to a toilet, such as, for example, a toilet seat or a ring. The sensing system 100 can be configured to measure one or more physical and/or physiological characteristics of users seated on the toilet seat. In some embodiments, the sensing system 100 can identify which user is seated on the toilet seat out of a plurality of different users based on physical and/or physiological characteristics measured of that user. The sensing system 100 includes one or more sensor(s) 110, a processor 120, a memory 122, and a communication interface 126.

The sensor(s) 110 can be configured to measure data that is indicative of one or more physical and/or physiological characteristics of a user. In some embodiments, the sensor(s) 110 can be coupled to or integrated with a toilet seat and/or disposed near a toilet, and be configured to measure data about a user when the user is seated on the toilet seat. Alternatively or additionally, the sensor(s) 110 can be coupled to, integrated with, or disposed near other lavatory devices, and be configured to measure data about a user when that user is using such devices and/or near such devices. The sensor(s) 110 can include one or more force sensor(s) 130, electrocardiogram (ECG) sensor(s) 140, and/or photoplethysmogram (PPG) sensor(s) 150. Optionally, in some embodiments, the sensor(s) 110 can also include other types of sensor(s) 160, as further described herein.

The force sensor(s) 130 of the sensing system 100 can be any suitable sensing device that can measure, record, and/or collect forces or loads, e.g., present on a toilet seat when a user is seated on the toilet seat. The measured forces can be used to determine one or more physical or physiological characteristics of the user. The force sensor(s) 130 can be disposed on, integrated, and/or coupled to a ring of a toilet (a toilet seat and/or other waste receptacle). For example, in some embodiments, a ring can include or be coupled to one or more supports (e.g., bumpers), which can be configured to accommodate the force sensor(s) 130 and support the ring on a top portion of a toilet bowl. Alternatively or additionally, the force sensor(s) 130 can be disposed in or adjacent to a hinge or coupler between the ring and a base of the toilet. The force sensor(s) 130 can include load cells (e.g., pneumatic load cells, hydraulic load cells, piezoelectric crystal load cells, inductive load cells, capacitive load cells, magneto strictive load cells, strain gauge load cells), strain gages, force sensing resistors (FSR) or printed or flexible force sensors, optical force sensors, etc.

The force sensor(s) 130 can be configured to measure force data that provides information regarding a weight or BCG of a seated user and/or subject e.g., by accounting for static and/or dynamic loads or forces present on the ring due to the weight of the subject seated on the ring of the toilet. The force sensor(s) 130 can be configured to measure changes in loads and/or forces, which can be used to calculate, for example, a weight change due to defecation or urination. In some embodiments, information collected by the force sensor(s) 130 can be used to determine the forces generated by the cardiac cycle of the seated user and/or subject. In particular, as the heart forcefully ejects fluid into the aorta of the user, the body of the user undergoes a downward and upward force in a repeating pattern, which can cause changes in forces and/or loads exerted by the user on the ring of the toilet. The force sensor(s) 130 can be configured to measure these changes and to provide BCG data for the user over time. In some embodiments, the force sensor(s) 130 can be independent sensors configured to each measure changes in forces and loads exerted by the user seated on the ring of the toilet and generate independent force data. The independent signals can be used to improve signal quality, which can enable more accurate and repeatable measurements.

In some embodiments, signals from multiple force sensor(s) 130 can be used to determine a seated posture of the user. For example, in some embodiments, the seat of the toilet can include a first number of force sensors 130 disposed on a front portion of the ring of the toilet (e.g., forward force sensors) and a second number of force sensors 130 disposed on the rear portion of the seat of the toilet (e.g., back force sensors). Higher relative signals on the forward force sensors can be indicative of the user leaning forward, with the ratio of the forward to back force sensors indicative of the posture angle. In some instances, the user weight, age gender, and/or a dimensional measurement associated with the user such as, for example, the user's height, a feet to waist length, or a seat to sternal notch distance can be used in conjunction with the static signals described above for a more accurate determination of posture.

In some embodiments, the force sensor(s) 130 described herein can be similar to the force sensor(s) described in U.S. Patent Application Publication No. 2022/0378373, incorporated above by reference.

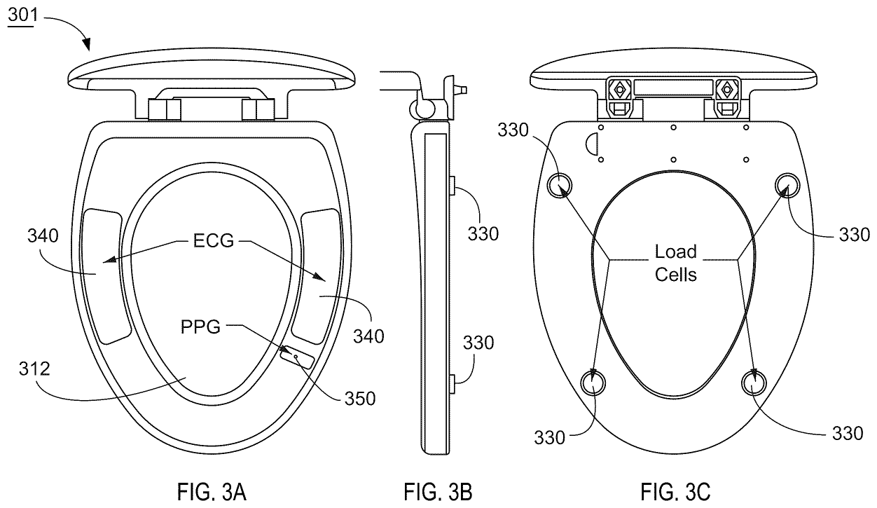

The ECG sensor(s) 140 of the sensing system 100 can be configured to measure an ECG of a subject, such as, for example, a user seated on a toilet. In particular, the ECG sensor(s) 140 can be configured to measure signals representative of the electrical activity of the heart of a user and/or subject originating from the depolarization of the conductive pathway of the heart and the cardiac muscle tissues during each cardiac cycle. In some embodiments, the ECG sensor(s) 140 can include one or more electrodes or conductive elements disposed on, integrated, and/or coupled to a surface of the seat of a toilet (or other waste receptacle). In some embodiments, the ECG sensor(s) 140 can include two or more electrodes or conductive elements for measuring a user's ECG. The electrodes can be disposed on or integrated into a toilet seat, as shown for example, in FIG. 3A, such that the electrodes can be placed in direct contact with skin or tissue of a user, when the user is seated on the toilet. In some embodiments, a processor (e.g., processor 120) operatively coupled to the ECG sensor(s) 140 can be configured to extract one or more features or characteristics from the ECG data, including, for example, heart rate, a R-peak amplitude, a PR interval, a QRS duration, a QT interval, etc.

The PPG sensor(s) 150 of the sensing system 100 can be any suitable sensing device that can be configured to measure a PPG of a subject, such as, for example, a user seated on a toilet. The PPG sensor(s) 150 can include an electromagnetic radiation source (e.g., a light source) that is configured to generate and direct an electromagnetic radiation signal (e.g., light signal) to a region and/or a tissue of a user, and the PPG sensor(s) 150 can include electromagnetic radiation sensors such as photodetectors, photodiodes, photoresistors, photovoltaic cells, etc. that can measure light reflected by, scattered, and/or transmitted through a region of skin or tissue of the user. The amount of light absorbed, reflected, scattered, and/or transmitted by the region of tissue can be associated with volumetric blood flow variations of the user. In some embodiments, the electromagnetic radiation source can include a light-emitting diode (LED), a Xenon Energy discharge lamp (XED), a fluorescent light source and or lamp, a mercury light source, an incandescent light source, a Laser diode, or the like. In some embodiments, the electromagnetic radiation source of the PPG sensor(s) 150 can be configured to generate electromagnetic radiation having one or more predetermined characteristics, such as, for example, a predetermined frequency or range thereof. For example, the electromagnetic radiation source of the PPG sensor(s) 150 can be configured to generate visible light, infrared (IR) light, ultraviolet (UV) light, etc. In some embodiments, the PPG sensor(s) 150 can include multiple electromagnetic radiation sources that can generate light having different characteristics. In some embodiments, the light source of the PPG sensor(s) 150 can be coupled to one or more filters and/or lenses designed to change, manipulate and/or precondition the electromagnetic radiation (e.g., light) generated by the light source. The PPG sensor(s) 150 can be disposed on, integrated, and/or coupled to the seat of a toilet (or other waste receptacle). In some embodiments, the PPG sensor(s) 150 can be disposed on or integrated into a toilet seat such that the PPG sensor(s) 150 can emit and capture reflected light or other electromagnetic radiation from a user, when that user is seated on the toilet seat.

In some embodiments, the PPG sensor(s) 150 described herein can be similar to the PPG sensor(s) described in International Patent Application No. PCT//US2023/075553, which is incorporated above by reference.

In some embodiments, the sensing system 100 can include one or more other types of sensors, such as sensor(s) 160. The sensor(s) 160 can be configured to measure one or more properties or characteristics of a user, a toilet or other lavatory device to which the sensor(s) 160 is coupled, and/or an environment near the user or toilet. In some embodiments, the measured data can be used to determine or evaluate one or more physical or physiological parameters and/or characteristics of a user.

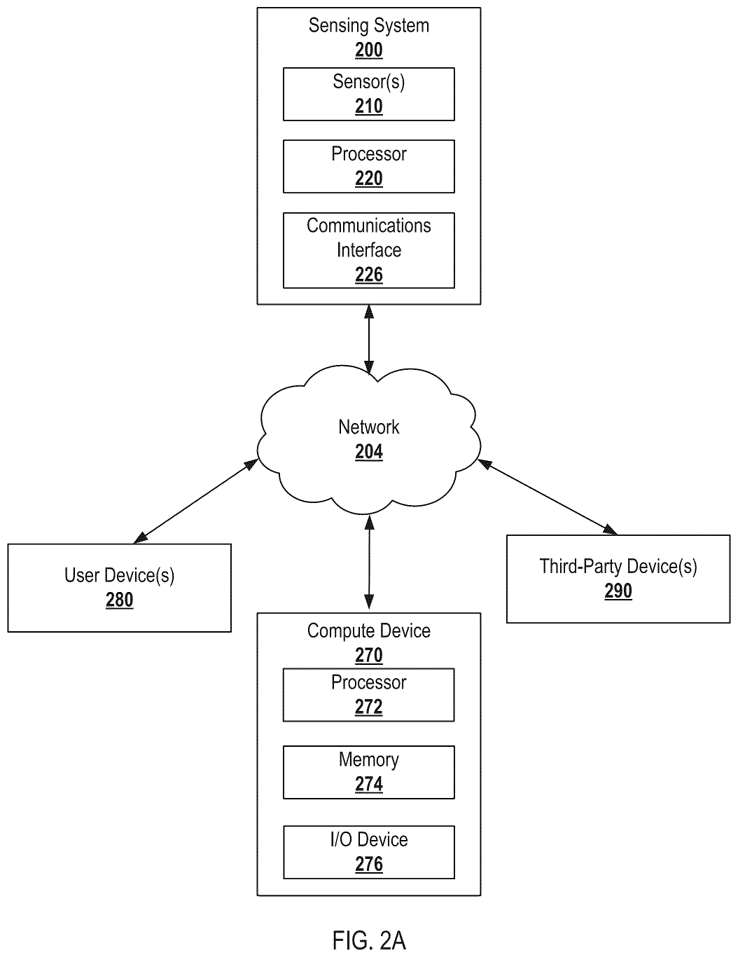

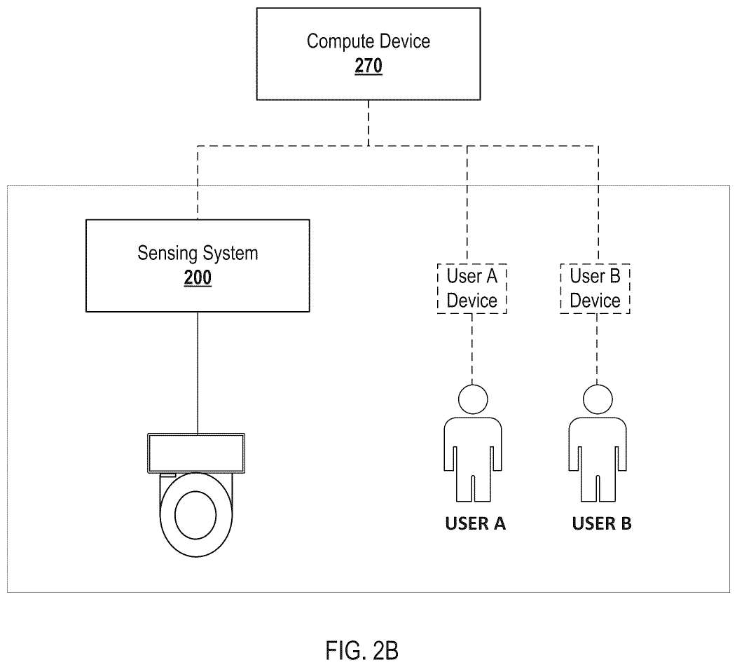

For example, in some embodiments, the sensor(s) 160 of the sensing system 100 can include a temperature sensor. The temperature sensor can be integrated and/or coupled to a toilet and configured to measure, record, and/or collect temperature of a urine stream or feces excreted by a user. In some embodiments, the temperature sensor can be coupled to a toilet seat. The housing can be sufficiently small such that the temperature sensor can fit within a toilet bowl (or other excretion collection device) without interfering with a user and/or individual's use of the toilet (or other excretion collection device). The temperature sensor may either be static or movable, e.g., in a linear motion and/or rotationally about an axis to span the opening of the toilet bowl (or other excretion collection device) through which urine and/or feces may be collected. As such, the temperature sensor can be used to capture a temperature of urine and/or feces regardless of the exact location where such urine and/or feces is received into the toilet bowl (or other excretion collection device). In an embodiment, the temperature sensor(s) can include one or more heat flux sensors integrated into the toilet seat to measure body core temperature. The heat flux sensor includes a thermal insulator disposed on a top surface of the toilet seat such that a first side and/or surface of the thermal insulator can be in direct contact with a portion of the skin of a user (e.g., a skin surface) when the user is seated on the toilet. The thermal insulator can be covered from a second side and/or surface by an electric heater that can be controlled and/or used to eliminate flow of heat through the insulator, until the temperature of the heater and the skin surface are equal (e.g., Zero heat flux conditions). At the zero heat flux conditions an isothermal tunnel is generated between the skin surface and subdermal tissue located a short distance below the skin surface, which approximates the temperature of the skin of the user (e.g., skin temperature). Additionally or alternatively, in some embodiments the thermal insulator can be coupled to a temperature sensor configured to measure and/or sense a temperature of the first side and/or surface of the thermal insulator, which facilitates measuring heat flux (e.g., by measuring the temperature of the first side and/or surface of the thermal insulator and the temperature of the heater) and skin temperature of the user (e.g., at Zero heat conditions). In some embodiments, the sensor(s) 160 of the system 100 can include one or more temperature and heat flux sensors integrated into a toilet seat, and operably coupled to the processor 120 and/or a processor of user device or a third-party device such as those described with reference to FIGS. 2A and 2B. The temperature and heat flux sensors can be used to sense and/or detect heat flux conditions, measure a temperature of a portion of the skin of a user (e.g., a skin surface) in direct contact with the heat flux sensors when the user is seated on the toilet. The measured temperature (or a signal associated with the measured temperature) and heat flux data can be transmitted to the processor 120 and be fed to a machine learning-based algorithm or an artificial intelligence model to determine a core body temperature of the user. In an embodiment, the temperature sensor(s) can include a non-contact infrared (IR) thermometer or temperature sensor, which can measure temperature based on the thermal radiation or black-body radiation emitted by the object being measured. In some embodiments, the temperature sensor described herein can be similar to the temperature sensors described in U.S. Patent Application Publication No. 2022/0361754, incorporated above by reference.

In some embodiments, the sensor(s) 160 of the sensing system 100 can include a camera and/or a spectrometer (with optional light source) integrated into a toilet seat such that the camera and/or spectrometer can be used in addition to the other sensors 110 to determine the identity of the user, or to gather additional physiological data such as a speckle plethysmogram. For example, in some embodiments the sensor(s) 160 can include a camera disposed and/or integrated on a surface of the toilet seat which is in direct contact with skin of a user when the user is seated on the toilet. The processor 120 can be configured to activate the camera to collect one or more images and determine, from the images, information associated with, for example, a tone and/or other characteristic of the skin of the user seated on the toilet. In some embodiments, the camera can be an infrared skin penetrating camera and/or spectrometer. The images collected by the camera can be used to determine the identity of the user seated on the toilet.

Additionally or alternatively, the sensor(s) 160 of the sensing system 100 can include two or more electrodes that function as impedance sensor(s). The impedance electrodes and/or sensors can be integrated and/or coupled to a surface of a toilet seat. In some embodiments the impedance sensor(s) can include two impedance sensors: a first impedance sensor disposed on a first side of the toilet seat (e.g., a left side or a right side), and a second impedance sensor disposed on a second side of the toilet seat opposite to the first side. The first and second impedance sensors can be disposed on the toilet seat such that the impedance sensors can be placed in direct contact with the buttocks (or any adjacent skin surface) of a user when the user is seated on the toilet (e.g., the first impedance sensor being in contact with a first buttock and the second impedance sensor being in contact with the second buttock). The two impedance sensors can be coupled such that the first and second impedance sensors can form a closed-loop circuit. The current and/or voltage associated with the closed-loop circuit can be measured (e.g., the voltage between the impedance sensors can be measured), processed (e.g., amplified, filtered, digitized) and send to a processor (e.g., processor 120). The first and second impedance sensors can be operatively coupled to a processor (e.g., the processor 120) that can receive signals from the first and second impedance sensors to determine a bioimpedance. When a user places his buttocks on the toilet seat (e.g., the left and right buttock in direct contact with the first and second impedance sensors), the first impedance sensor and/or electrode can send a signal (e.g., a current) to the second impedance sensor and/or electrode, and a resulting voltage can be measured between the two electrodes to determine a buttock-to buttock bioimpedance of the user.

In some embodiments, the sensor(s) 160 of the sensing system 100 can include three electrodes that can be configured to function as impedance sensors or as ECG sensors. For example, the three electrodes can be configured as a 3-electrode system. The 3-electrode system can include a first and a second electrode which can be and/or function as single lead signal ECG electrodes; and a third electrode which can be a driven-right-leg (DRL) electrode that functions as a reference point for the ECG signal(s). The first, second, and third electrodes can be operatively coupled (e.g., via an onboard chip or integrated circuit) to a processor (e.g., the processor 120) that can determine ECG data and/or a bioimpedance of a user when the user is seated on the toilet. The onboard chip can be an auxiliary processor configured to receive raw ECG data from the first, second, and third electrode, conduct one or more pre-processing steps to the raw ECG data (e.g., amplify, filter, and/or digitize the raw ECG data recorded by the first, second and third electrode) and then send the pre-processed ECG data to the processor 120. In some embodiments, the onboard chip can be a multi-channel ECG chip.

In some embodiments, the sensor(s) 160 of the sensing system 100 can include four electrodes that function as impedance sensor(s). The electrodes and/or sensors can include: a first and a second impedance sensor disposed on a first side of the toilet seat (e.g., a left side or a right side), and a third and a fourth impedance sensor disposed on a second side of the toilet seat opposite to the first side. The first and second impedance sensors can be disposed on the toilet seat such that the first and second impedance sensors can be placed in direct contact with a buttock (or any adjacent skin surface including for example a thigh) of a user when the user is seated on the toilet (e.g., the first and the second impedance sensor being in contact with a first buttock of the user). The third and fourth impedance sensor can be disposed on the toilet seat such that the third and fourth impedance sensors can be placed in direct contact with the other buttock (or an adjacent skin surface including for example a thigh) of the user when the user is seated on the toilet (e.g., the third and the fourth impedance sensors being in contact with the second buttock of the user). The four impedance sensors can be coupled such that the first, second, third, and fourth impedance sensor form and/or constitute a kelvin-like drive circuit and/or a four-electrode configuration. The kelvin-like drive circuit configuration can facilitate eliminating resistive voltage losses and/or drops caused by the current source, resulting in more accurate impedance measurement. Additionally, the kelvin-like drive circuit can exhibit reduced sensitivity to the specific characteristics and geometry of the electrical connections, as well as to contact impedance (or a changing contact impedance which would cause your measured impedance to drift over time)

In some embodiments, the first and the third impedance sensors (e.g., one impedance sensor disposed on each side of the toilet seat) can be configured to act and/or operate as excitation electrodes, which deliver an excitation signal (e.g., apply an excitation current trough the first and the third impedance sensors) when the user is seated on the toilet. The second and the fourth impedance sensors (one impedance sensor disposed on each side of the toilet seat) can be configured to act and/or operate as sensing electrodes, which sense and/or measure a response signal in response to the excitation signal (e.g., measure a voltage difference, loss, and/or drop across the first and the third impedance sensor caused by the excitation current). The first, second, third, and fourth impedance sensor can be operably coupled to a processor (e.g., the processor 120) that can receive signals from the first, second, third, and fourth impedance sensors to determine a buttock-to-buttock bioimpedance of the user.

Additionally and/or alternatively, in some embodiments, the first and the second impedance sensors (e.g., the two impedance sensors disposed on a side of the toilet seat, either the first side or the second side of the toilet seat) can disposed such that tissue of a thigh of the user seated on the toilet can be placed in contact with the first and the second impedance sensors. That is to say, a first portion of a thigh of the user can be placed in direct contact the first impedance sensor and/or electrode while a second portion of that thigh of the user can be placed in direct contact with the second impedance sensor and/or electrode. The first and second impedance sensors can be coupled such that the first and second impedance sensors can form a closed-loop circuit. The current and/or voltage associated with the closed-loop circuit can be measured (e.g., the voltage between the impedance sensors can be measured), processed (e.g., amplified, filtered, digitized) and send to a processor (e.g., processor 120). As described above, the processor can be configured to determine a bioimpedance based on the measured current and/or voltage. When a user places one of the user's thigh (a left or a right thigh of the user) in contact with the first and second impedance sensor disposed on the toilet seat such that a first portion of the thigh of the user placed in contact with the first impedance sensor and a second portion of that thigh of the user in contact with the second impedance sensor, the first impedance sensor can send a signal (e.g., a current) to the second impedance sensor, and a resulting voltage can be measured between the first and the second impedance sensor to determine a bioimpedance of the thigh of the user. Similarly, the third and fourth impedance sensors can be disposed such that tissue of a thigh of the user seated on the toilet can be placed in contact with the third and the fourth impedance sensors, and a bioimpedance of the thigh of the user can be measured. In some embodiments the sensor(s) 160 of the sensing system 100 can be configured to determine a buttock-to-buttock bioimpedance of the user as well as a thigh impedance of the user.

In some embodiments, the impedance sensors and/or electrodes described above can be configured to generate an excitation signal and measure a response signal to determine a bioimpedance of the user at a predetermined frequency. In some embodiments the impedance electrodes and/or sensors described above can be configured to determine a bioimpedance of the user at multiple frequencies. For example, in some embodiments the impedance electrodes and/or sensors can be configured to determine a bioimpedance of a user at multiple frequencies between 40 Hz and 10 MHz. In some embodiments, the impedance electrodes and/or sensors can be configured to determine bioimpedance data of the user sequentially (e.g., one frequency at a time producing a frequency sweep). Alternatively, the impedance electrodes and/or sensors can be configured to determine bioimpedance data of the user at different frequencies simultaneously. In some embodiments the impedance electrodes and/or sensors can be configured to determine bioimpedance data of the user continuously while the user is seated on the toilet. In other embodiments the impedance electrodes and/or sensors can be configured to determine bioimpedance data of the user throughout random periods of time while the user is seated on the toilet.

In some embodiments, the force sensor(s) 130, ECG sensor(s) 140, PPG sensor(s) 150 and/or other sensors can be disposed in spaced relation to one another, e.g., at different locations along a toilet ring. In some embodiments, one or more sensors can be disposed adjacent to or near a toilet ring or other component of a toilet, while other sensors are disposed on the toilet ring or other toilet component. For example, one or more force sensors in a scale can be disposed adjacent to a toilet, while other force sensors can be disposed in the toilet (e.g., in a ring or a coupler attached to the ring). In some embodiments, one or more of the force sensor(s) 130, ECG sensor(s) 140, and/or PPG sensor(s) 150 can be disposed together on a single circuit board or housing.

In some embodiments, the data measured, recorded, and/or collected by one or more of the sensor(s) 110 can be used alone or in combination with other information to identify a user, e.g., when the user is seated on a toilet. Because individual users tend to have physiological characteristics that are different from that of other users, the sensing system 100 can be configured to analyze data indicative of the physiological characteristics of a user to identify that user. In some embodiments, the sensor(s) 130, 140, 150, 160 can collectively measure data associated with a plurality of physiological characteristics of a user and provide this measured data to a processor (e.g., processor 120 or a processor of a separate compute device (see FIGS. 2A-2B)). The processor 120 can be any suitable processing device configured to run and/or execute a set of instructions or code. The processor 120 may be, for example, a general purpose processor, a Field Programmable Gate Array (FPGA), an Application Specific Integrated Circuit (ASIC), a Digital Signal Processor (DSP), and/or the like. The processor 120 may be configured to run and/or execute application processes and/or other modules, processes and/or functions associated with the system 100 and/or a network associated therewith (see FIGS. 2A-2B).

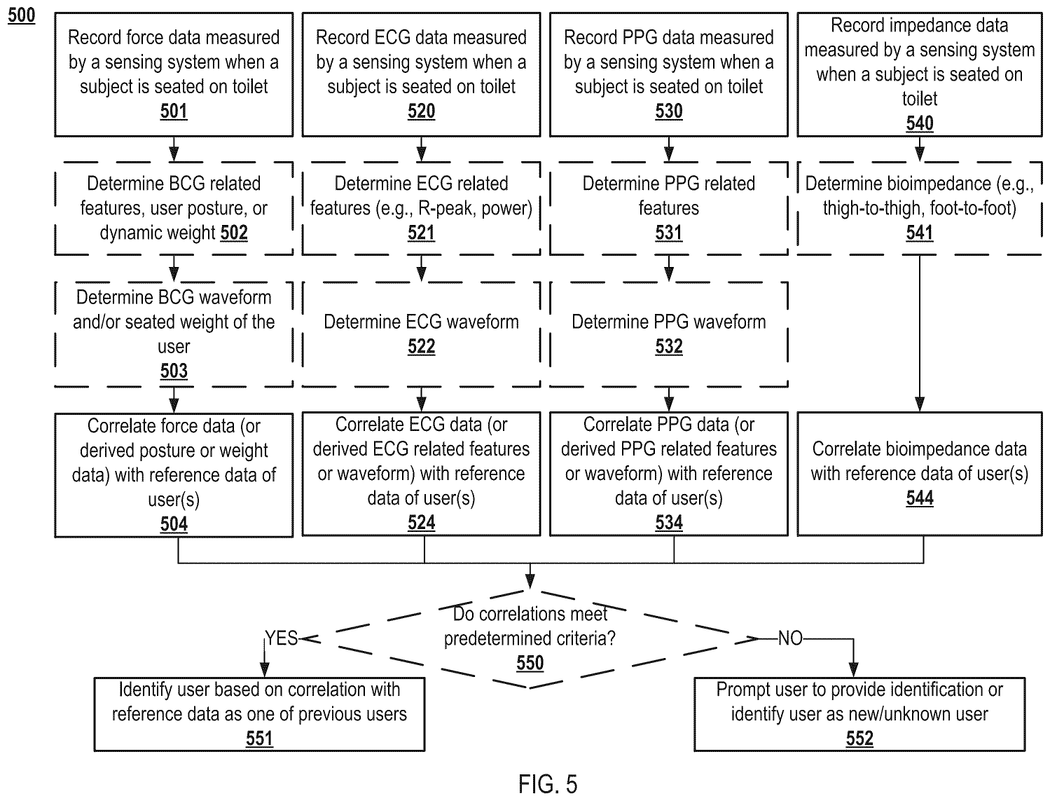

In some embodiments, the processor 120 can be configured to analyze data measured by one or more sensor(s) 110 to identify a user. For example, the processor can be configured to process the measured data, e.g., to remove noise, artifacts, and/or other compromised data. The processor can be configured to extract one of more features from the measured data and/or to determine one or more physiological characteristics of the user (e.g., core body temperature, weight, BCG, ECG, posture, impedance, or other physiological parameters and/or characteristics) based on the measured data. The processor can be configured to compare the measured data (or extracted features of the data) with reference data (e.g., previously measured or stored data of one or more users) to determine whether the measured data is similar to the reference data. Based on the comparison, the processor may be able to identify the user associated with the measured data. For example, if the measured data is substantially similar to a reference data set associated with User A, then the processor can determine that the measured data is of User A. In some instances, where the measured data may not be similar to any reference data, then the processor may indicate that the user associated with the measured data is a new user. Further details of this process are provided below with reference to FIG. 5.

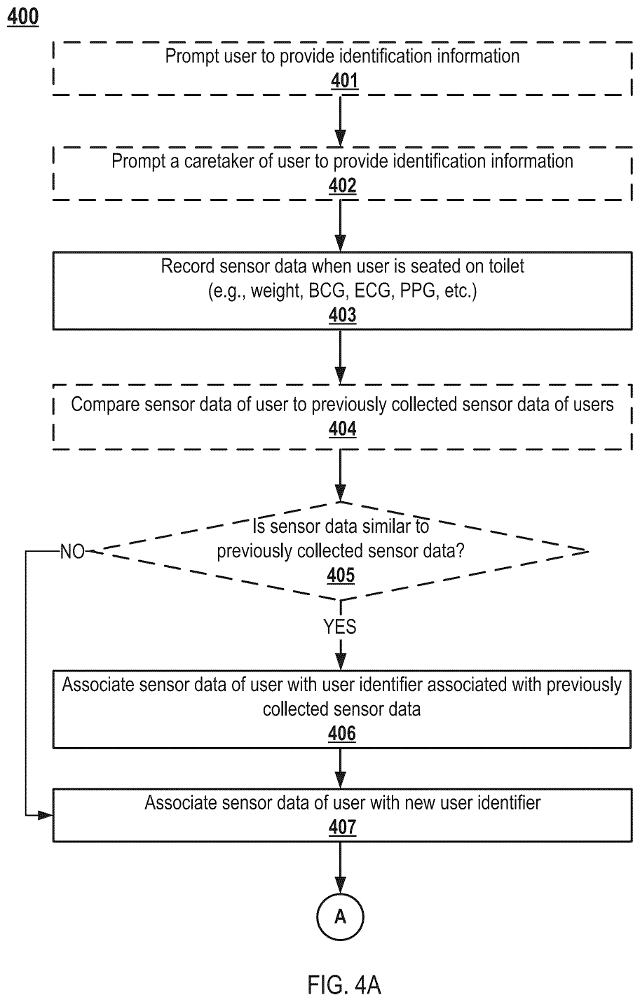

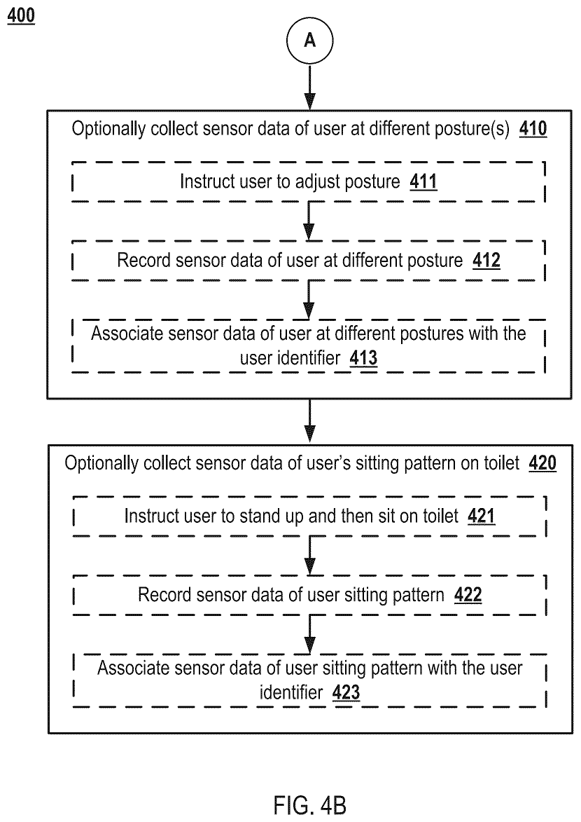

In some embodiments, the system 100 may implement onboarding of new users. For example, a new user of a toilet may indicate via an input/output device (e.g., an input/output device coupled to communications interface 126) that the user is a new user. The system 100, via the one or more sensor(s) 110, can record sensor data of the user when the user is seated on the toilet, including, for example, core body temperature, weight, BCG, ECG, posture, impedance, or other physiological parameters and/or characteristics. The system can then associate recorded sensor data with the new user, and store the recorded sensor data as reference data for identifying that user during that user's subsequent uses of the toilet. Further details of this process are provided below with reference to FIGS. 4A-4B.

In some embodiments, the processor 120 can be configured to analyze data measured by one or more sensor(s) 110 to monitor and/or evaluate various physiological data or conditions of the subject. For example, the processor can be configured to process and/or analyze sensor data (e.g., received from the force sensor(s) 130, the ECG sensor(s) 140, the PPG sensor(s) 150, and/or the other sensor(s) 160) to determine a temperature, weight, BCG, ECG, posture, impedance, or other physiological parameters and/or characteristics of an individual or subject. The processor can be configured to monitor these physiological characteristics of the user and/or compare them to predetermined metrics associated with certain conditions. The processor can inform the user (or the user's healthcare providers or caretakers) of changes in such data or conditions necessitating certain therapies, treatments, lifestyle changes, etc. Examples of processing and/or evaluating sensor data to determine physiological parameters and/or characteristics about a user and/or subject are described in U.S. Pat. No. 10,292,658, U.S. Patent Application Publication No. 2022/0378373, U.S. Patent Application Publication No. 2022/0364904, U.S. Patent Application Publication No. 2022/0361754, and International Patent Application No. PCT/US2023/075553, each of which is incorporated above by reference.

The processor 120 can be coupled to the communication interface 126, which can be used to send information to and/or receive information from other devices, as further described herein. The communication interface can be configured to allow two-way communication with an external device, including, for example, the compute device 270, one or more user device(s) 280, and/or one or more third-party device(s) 290, as depicted in FIGS. 2A-2B. The communication interface can include a wired or wireless interface for communicating over a network (e.g., the network 204).

The processor 120 can be operatively coupled to the memory 122. The memory 122 can be, for example, a random-access memory (RAM), a memory buffer, a hard drive, a database, an erasable programmable read-only memory (EPROM), an electrically erasable read-only memory (EEPROM), a read-only memory (ROM), and/or so forth. In some embodiments, the memory 122 stores instructions that cause processor 120 to execute modules, processes, and/or functions associated with processing and/or analyzing sensor data from sensor(s) 110, identifying a subject seated on the toilet based on data measured, recorded, and/or collected by the sensor(s) 110, and/or sending sensor data to other devices via communications interface 126.

FIG. 2A depicts a block diagram illustrating a sensing system 200 in communication with other devices via a network 204, according to embodiments. In some embodiments, the sensing system 200 can be configured to measure physiological data and/or characteristics of a subject seated on a toilet or other lavatory device. For example, the sensing system 200 can be configured to measure one or more of load or force data indicative or a weight or seated weight, BCG data, ECG data, PPG data, core body temperature, bioimpedance, or the like. In some embodiments, the sensing system 200 can be operatively coupled to one or more of a compute device 270, a user device 280, or a third-party device 290, and to send the measured data to such devices for further processing and/or analysis. In some embodiments, the sensing system 200 or one or more devices coupled to the sensing system (e.g., a compute device 270, a user device 280, or a third-party device 290) can be configured to associate the data measured by the sensing system 200 with a particular individual or subject. In some embodiments, the sensing system 200 or one or more devices coupled to the sensing system (e.g., a compute device 270, a user device 280, or a third-party device 290) can be configured to identify an individual or subject based on the data measured by the sensing system 200.

The sensing system 200 can include component(s) that are structurally and/or functionally similar to those of other sensing systems and devices described herein, including, for example, sensing system 100. For example, sensing system 200 can have one or more sensors 210. The sensor(s) 210 can include force sensor(s), ECG sensor(s), PPG sensor(s), and/or other sensor(s), similar to those described with reference to sensing system 100. The sensor(s) 210 can be disposed on, integrated, and/or coupled to a seat of a toilet to measure sensor data representative of physiological parameters and/or characteristics about a subject.

In some embodiments, the sensing system 200 can be configured to communicate with a compute device 270, one or more user device(s) 280, and/or one or more third-party device(s) 290, via a network 204. The network 204 can include one or more network(s) that may be any type of network (e.g., a local area network (LAN), a wide area network (WAN), a virtual network, a telecommunications network) implemented as a wired network and/or wireless network and used to operatively couple to any compute device, including the sensing system 200, the compute device 270, the user device(s) 280, and the third-party device(s) 290.

In some embodiments, the sensing system 200 can be configured to send data measured by the sensor(s) 210 to the compute device 270, one or more user device(s) 280, and/or one or more third-party device(s) 290, via a communication interface 226. In some embodiments, the sensing system 200 can include onboard processing, such as, for example, a processor 220 implemented as a microprocessor, to process (e.g., filter, convert, etc.) sensor data prior to sending the sensor data to the compute device 270, one or more user device(s) 280, and/or one or more third-party device(s) 290. Alternatively, the sensing system 200 can be configured to send raw sensor data to the compute device 270, one or more user device(s) 280, and/or one or more third- party device(s) 290. In some embodiments, the processor 220 can be configured to analyze the data measured by the sensor(s) 210 and/or determine information such as a seated weight, BCG data, ECG data, PPG data, or other physiological parameters and/or characteristics of a subject seated on a toilet. In some embodiments, the sensing system 200 can be configured to receive information from a user device 280, which can be used to determine the identity of the user. For example, in some instances, an unknow user can be seated on the toilet while having with him a user device 280. The user device 280 can include a mobile phone or other portable device, a wearable device such as a necklace, a ring, or the like, a tablet, a laptop, a personal computer, a smart device, etc. The user device 280 can be configured to communicate with the compute device 270 via the network 204 to send information and/or data of the user such that the compute device can determine the identity of the user seated on the toilet. The sensing system 200 can be configured to receive from the compute device 270 the identity of the user seated on the toilet. Additionally or alternatively, in some embodiments the user device 280 can be configured to communicate with the processor 220 of the sensing system 200 to send information and/or data of the user. The sensing system 200 can use the received information and/or data from the user device 280 to determine the identity of the user seated on the toilet.

In some embodiments, the processor 220 can be configured to associate the measured data and/or physiological parameters with a particular individual or subject. In some embodiments, the measured data and/or physiological parameters can be stored as reference data in a memory of the sensing system 200 (not shown). Alternatively, in some embodiments, the measured data and/or physiological parameters can be stored as reference data in a memory or database coupled to the sensing system 200, such as a memory of an external compute device (e.g., compute device 270, user device(s) 280, third-party device(s) 290, and/or other compute device). In some embodiments, the processor 220 can be configured to store reference data of a plurality of known subjects (i.e., subject with known identities), where the reference data of each subject is associated with that subject and can be used to uniquely identify that subject. In particular, the processor 220 of the sensing system 200 can be configured to receive new data measured by the sensor(s) 210 of an unknown subject seated on the toilet and to analyze the new data to determine an identity of the unknown subject. For example, the processor 220 can be configured to compare the new data (or information determined or extracted from the new data) with the data and/or physiological parameters previously stored (e.g., the reference data) to determine whether the unknown subject is one of the plurality of subjects with which the processor 220 is familiar. In instances where the processor 220 determines that there is a sufficient degree of similarity or overlap between the new measured data of the unknown subject and the reference data of a known subject, the processor 220 can determine that the unknown subject and the known subject have the same identity. For example, the processor 220 can perform cluster analysis or clustering and determine that the new data of the unknown subject belongs to the same cluster as the data associated with one of the known subjects. When the processor 220 can identify the unknown subject based on the comparison, the processor 220 can associate the new measured data with the identity of the known subject and store such data as future reference data (e.g., in an onboard memory or an external memory). In instances where the processor 220 cannot identify the unknown subject (e.g., due to lack of similarity with data of a known subject), the processor 220 can indicate that the unknown subject cannot be identified and/or prompt the unknown subject to provide identifying information. Furthermore, in some embodiments when the new data measured by the sensor(s) 210 of an unknown subject seated on the toilet cannot lead to identifying the user, the sensing system 200 may be configured to store this new data measured in a memory of the sensing system 200, the memory 274 of the compute device 270, and/or a memory of a third-party device 290 as measured data of unrecognized users. In instances where the processor 220 receives new data measured by the sensor(s) 210 of an unknown subject, the processor can be configured to compare the new data (or information determined or extracted from the new data) with the data and/or physiological parameters previously stored (e.g., the reference data), as well as with the data of the unrecognized users. When the processor 220 can identify the unknown subject based on a comparison with data of unrecognized users, the processor can be configured prompt the unrecognize user to to provide identifying information.

In some embodiments, the processor 220 can be configured to present and/or communicate to a subject the data measured and analyzed by the sensor(s) 210 and/or information extracted from that data (e.g., the seated weight, BCG data, ECG data, PPG data, or other physiological parameters and/or characteristics) via an onboard display, audio device, or other output device. In some embodiments, the processor 220 can be configured to present and/or communicate to the subject the determined identity of the subject seated on a toilet. In some embodiments, the processor 220 can be configured to present and/or communicate to the subject that the processor was unable to identify the subject and/or prompt the subject to provide identifying information.

While the functions and processes described above are described with reference to the processor 220 of the sensing system 200, in some embodiments, such functions and processes can wholly or partially be performed or implemented by a separate compute device (e.g., compute device 270, user device(s) 280, and/or third-party device(s) 290). In particular, in some embodiments, the processor 220 can be configured to send the data measured by the sensor(s) 210 (or information extracted or determined from the data measured by the sensor(s) 210) to an external device, e.g., via the communication interface 226. The communication interface 226 can be configured to allow two-way communication with one or more external devices, including, for example, the compute device 270, one or more user device(s) 280, and/or one or more third-party device(s) 290. The communication interface 226 can include a wired or wireless interface for communicating over the network 204.

The compute device 270 can be configured to perform a variety of processing tasks, including, for example, processing and/or analyzing the sensor data measured by the sensor(s) 210 (or information extracted or determined from the data measured by the sensor(s) 210), determining an identity of an unknown subject based on the measured sensor data (or information extracted or determined from the measured sensor data), etc. The compute device 270 can include a processor 272, a memory 274, and an input/out device (I/O) 247 (or a multiplicity of such components).

The memory 274 can be, for example, a random-access memory (RAM), a memory buffer, a hard drive, a database, an erasable programmable read-only memory (EPROM), an electrically erasable read-only memory (EEPROM), a read-only memory (ROM), and/or so forth. In some embodiments, the memory 274 stores instructions that cause processor 272 to execute modules, processes, and/or functions associated with processing and/or analyzing sensor data from sensing system 200, identifying a subject seated on the toilet, etc.

The processor 272 of compute device 270 can be any suitable processing device that can execute the modules, processes, and/or functions stored in the memory 274. For example, the processor 272 can be configured to process and/or analyze sensor data (e.g., measured by sensor(s) 210), to determine a weight, BCG, or other physiological data or conditions of an individual. The processor 272 can be configured to associate the sensor data and/or determined physiological data or conditions with a particular individual or subject and to store this data as reference data. The processor 272 can be configured to identify unknown subjects based on sensor data, e.g., by comparing the measured data of the unknown subject with the reference data. The processor 272 can be a general-purpose processor, a Field Programmable Gate Array (FPGA), an Application Specific Integrated Circuit (ASIC), a Digital Signal Processor (DSP), and/or the like.

The I/O device 276 of the compute device 270 can include one or more components (e.g., a communication or network interface) for receiving information and/or sending information to other devices (e.g., sensing system 200, user device(s) 280, third-party device(s) 290). In some embodiments, the I/O device 276 can optionally include or be operatively coupled to a display, audio device, or other output device for presenting information and/or communicating with a user. For example, in some embodiments the I/O device 276 can include a vibration motor disposed in the toilet seat to enable haptic communication with a user. In some embodiments, the I/O device 276 can optionally include or be operatively coupled to a touchscreen, a keyboard, or other input device or receiving information from a user.

In some embodiments, the compute device 270 can be a nearby compute device (e.g., a local computer, laptop, mobile device, tablet, etc.) that includes software and/or hardware for receiving the sensor data and processing and/or analyzing the sensor data. In some embodiments, the compute device 270 can be a server that is remote from the sensing system 200 but can communicate with the sensing system 200 via the network 204 and/or via another device on the network 204 (e.g., a user device 280). For example, the sensing system 200 can be configured to transmit sensor data to a nearby device (e.g., a user device 280), e.g., via a wireless network (e.g., Wi-Fi, Bluetooth®, Bluetooth® low energy, a 2G, 3G, 4G/LTE, 5G, and/or other cellular network, Zigbee and the like), and then that device can be configured to transmit the sensor data to the compute device 270 for further processing and/or analysis.

The user device(s) 280 can be compute device(s) that are associated with a user or subject that uses a toilet or other lavatory device equipped with the sensing system 200. Examples of user device(s) 280 can include a mobile phone or other portable device, a wearable device such as a necklace, a ring, or the like, a tablet, a laptop, a personal computer, a smart device, etc. In some embodiments, a user device 280 can receive sensor data from the sensing system 200 and process that sensor data before passing the sensor data to the compute device 270. For example, a user device 280 can be configured to reduce noise (e.g., filter, time average, etc.) raw sensor data. In some embodiments, a user device 280 can be configured to analyze the sensor data and present (e.g., via a display) information representative of or summarizing the sensor data, a determined identity of the subject, and/or other information (e.g., a confidence of the determined identity, alerts for failing to determine the identity of a user, alerts for detecting significant changes in the physiological data of a user, etc.). For example, the user device 280 can present weight information, ECG information, BCG information, bioimpedance information, etc. to a user. In some embodiments, a user device 280 can transmit the sensor data to the compute device 270 or other external devices, which can analyze the sensor data and send information representative of or summarizing the sensor data or identifying the user back to the user device 280 for presenting (e.g., via a display) to a user. For example, in some embodiments the user device 280 can present to a user information about the user such as heart rate, heart rate variability, left ventricular ejection time, pre-ejection period, flow velocity, pulse transit time (e.g., based on ECG or BCG data), blood pressure, cardiac output, cardiac contractility, abnormal heart function, blood oxygenation levels (e.g., SpO₂), respiration rate, stress levels (e.g., via heart rate variability), body weight, and/or cardiac waveform characteristics (e.g., magnitudes and/or intervals).

The third-party device(s) 290 can be compute device(s) associated with other individuals or entities that have requested and/or been provided access to a user's data. For example, the third-party device(s) 290 can be associated with healthcare professionals (e.g., physicians, nurses, therapists) and/or caregivers of the user. The user can select to have certain third parties have access to the user's health data (e.g., including health data obtained from sensor data collected by sensing system 200). The third parties can then track the user's health information to determine whether the user is at risk for certain conditions and/or needs certain interventions, treatments, or care.

While the user device(s) 280, and third-party-device(s) 290 are not depicted with any onboard memory, processing, and/or I/O devices, it can be appreciated that any one of these devices can include components (e.g., a memory, a processor, a I/O device, etc.) that enable it to perform functions such as, for example, processing and/or analyzing the sensor data, or using the sensor data to determine physiological information about an individual, and/or identifying an individual based on the determined physiological information (e.g., seated weight, ECG, PPG, BCG, body core temperature, posture, impedance, etc.).

FIG. 2B schematically illustrates an instance in which the sensing system 200 is placed in communication with the compute device 270 to identify multiple unknown and/or unidentified users based on data measured by the sensing system 200. More specifically, FIG. 2B depicts a room where there is a first unknown user (e.g., user A) optionally associated with a first user device 280 (e.g., user A device), a second unknown user (user B) optionally associated with a second user device 280 (e.g., user B device), and a toilet equipped with the sensing system 200. Although not shown in FIG. 2B, the sensing system 200 includes the sensor(s) 210, the processor 220, and the communications interface 226 described above with reference to FIG. 2A. The sensing system 200 can measure new sensor data of one of the unknown user via the sensor(s) 210 when said unknown user is seated on the toilet (e.g., user A or user B). The sensing system 200 can be configured to send the new sensor data measured by the sensor(s) 210 to the compute device 270, and/or one or more third-party device(s) 290, via the communication interface 226. In some embodiments, the processor 220 can be configured to pre-process (e.g., filter, convert, etc.) the new sensor data prior to sending the new sensor data to the compute device 270, and/or one or more third-party device(s) 290. Alternatively, in some embodiments the sensing system 200 can be configured to send raw new sensor data to the compute device 270, and/or one or more third-party device(s) 290. The compute device 270 can be configured receive the new sensor data and analyze the new sensor data to determine an identity of the unknow user. For example, in some embodiments the compute device 270 can be configured to receive the new sensor data measured by the sensor(s) 210 and determine information such as a seated weight, BCG data, ECG data, PPG data, bioimpedance, or other physiological parameters and/or characteristics of the unknow user seated on the toilet. The compute device 270 can be further configured to compare the new sensor data (or information determined or extracted from the new sensor data) with data and/or physiological parameters of a plurality of user previously stored (e.g., the reference data) either in the memory 274 of the compute device 270, or in a memory of a third-party device 290. The compute device 270 can compare the new sensor data with the reference data to determine whether the unknown user seated on the toilet is one of the plurality of users with which the compute device 270 is familiar. In instances where the compute device 270 determines that there is a sufficient degree of similarity or overlap between the new sensor data of the unknown user and the reference data of a known user, the compute device 270 can determine that the unknown user and the known subject have the same identity. That is to say, the compute device 270 can determine whether the unknown user seated on the toilet is either user A or user B. Optionally, in some embodiments the processor 220 of the sensing system 200 can be configured to receive the new sensor data measured by the sensor(s) 210 and analyze the new sensor data to determine an identity of the unknown user as described above.

FIG. 2B shows the compute device 270 can optionally be coupled to multiple user devices 280 such as the user A device and/or to the user B device. In such embodiments, the processor 272 of the compute device 270 can be configured to determine whether the unknown user seated on the toilet is either user A or user B, and then communicate via the network 204 with the user device 280 of the identified user (e.g., user A device or user B device) and prompt the identified user to confirm the user's identity. Alternatively, in some embodiments the processor 220 of the sensing system 200 can be configured to communicate via the network 204 with the user device 280 of the identified user (e.g., user A device or user B device) and prompt the identified user to confirm the user's identity.

FIGS. 3A-3C show a top, side, and bottom view, respectively, of a sensing device or system (e.g., such as those described with reference to FIGS. 1 and 2) for monitoring signals associated with various physiological data or conditions of an individual, according to embodiments. As shown in FIGS. 3A-3C, the sensing device is implemented as a toilet seat 301. The toilet seat 301 can be a toilet ring or other component of a toilet on which an individual or subject sits. The toilet seat 301 can be positioned on top of a base (e.g., a top portion of a toilet bowl), such that the toilet seat 301 defines a centrally disposed opening 312 therethrough, e.g., for receiving bodily fluids, defecation, etc. The shape and/or the dimensions of the toilet seat 301 can be circular, oval, elliptical, and/or any other closed annular shape (e.g., a round or “o” shaped toilet seat). Alternatively, in some embodiments the shape and/or dimensions of the toilet seat 301 can correspond to an open shape such as, for example, a “U” shape toilet seat (not shown in FIGS. 3A-3C. In such embodiments, the toilet seat 901 can include one or more openings that provide and/or generate spaces and/or gaps between the seat and the bowl of the toilet that facilitate a user to sit on the toilet without directly contacting the bowl with their private parts, particularly in the case of male users. For example, in some embodiments the toilet seat 901 can include an opening disposed on a front portion of the toilet seat 901. This opening, which can also be referred to as a front opening of the toilet 901, can create a place where urine may splash, facilitating rapid cleaning of the toilet when the toilet is installed in, for example, a public washroom and/or bathroom.

The toilet seat 301 can include multiple sensors including, for example, one or more force sensor(s) 330, an ECG sensor 340, and a PPG sensor 350. The sensor(s) 310 can be the same or similar in form and/or function to the sensor(s) 110 and 210 described above with reference to FIGS. 1 and 2. The sensor(s) 310 included and/or incorporated in the toilet seat 301 can be configured to measure multiple signals (e.g., PPG signals, ECG signals, and loads and/or forces) present on the toilet seat 301, e.g., when an individual is seated on the toilet seat 301. While not depicted, the toilet seat 301 can also include a processor, a memory, and a communication interface, similar to those described with reference to FIGS. 1 and 2. Alternatively, the toilet seat 301 can be operatively coupled to a processor, a memory, and/or a communication interface that is not located in the toilet seat, such as that disposed within another component of the toilet (e.g., a base of the toilet) or disposed in an external device (e.g., compute device 270). In some embodiments, the toilet seat 301 can be configured to transmit sensor data collected by the force sensor(s) 330, ECG sensor 340, and PPG sensor 350 to a processor (e.g., processor 120, 220, 272), such that the processor can process and/or analyze the sensor data, as described above with reference to FIG. 2. In some embodiments, the sensing device depicted in FIG. 3 can be installed on an existing toilet, e.g., by retrofitting an existing toilet.

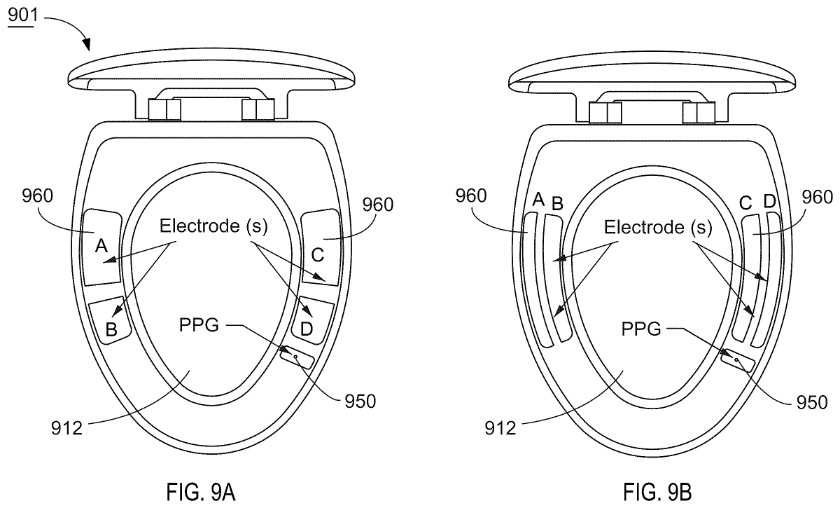

FIGS. 9A and 9B show the top view of two example sensing devices or systems (e.g., such as those described with reference to FIGS. 1, 2A-2B, and 3A-3C) for monitoring signals associated with various physiological data or conditions of an individual, according to embodiments of the present disclosure. As shown in FIGS. 9A and 9B, the sensing devices are implemented as a toilet seat 901. The toilet seat 901 can be a toilet ring or other component of a toilet on which an individual or subject sits. The toilet seat 901 can be positioned on top of a base (e.g., a top portion of a toilet bowl), such that the toilet seat 901 defines a centrally disposed opening 912 therethrough, e.g., for receiving bodily fluids, defecation, etc. The shape and/or the dimensions of the toilet seat 901 can be circular, oval, elliptical, and/or any other closed annular shape (e.g., a round or “o” shaped toilet seat). As described above, in some embodiments the shape and/or dimensions of the toilet seat 901 can correspond to an open shape such as, for example, a “U” shape toilet seat. For example, in some embodiments the toilet seat 901 can include an opening disposed on a front portion of the toilet seat 901. This opening, which can also be referred to as a front opening of the toilet 901, can create a place where urine may splash, facilitating rapid cleaning of the toilet when the toilet is installed in, for example, a public washroom and/or bathroom.

The toilet seat 901 can include multiple sensor(s) 910 including a PPG sensor 950, and four sensors 960 which can be four electrodes. The four sensors and/or electrodes 960 can be configured to function as impedance sensor(s) and/or ECG sensor(s). The PPG sensor 950 shown in FIGS. 9A and 9B can be the same or similar in form and/or function to the PPG sensors 150 and 350, described above with reference to FIGS. 1, and 3A, respectively. Additionally or alternatively, in some embodiments the sensor(s) 910 can also include force sensors configured to record, and/or collect forces or loads, e.g., present on the toilet (not shown in FIGS. 9A-9B). The sensor(s) 910 included and/or incorporated in the toilet seat 901 can be configured to measure multiple signals (e.g., PPG signals, bioimpedance signals, ECG signals, and loads and/or forces) present on the toilet seat 901, e.g., when an individual is seated on the toilet seat 901. While not depicted, the toilet seat 901 can also include a processor, a memory, and a communication interface, similar to those described with reference to FIGS. 1, and 2A-2B. Alternatively, the toilet seat 901 can be operatively coupled to a processor, a memory, and/or a communication interface that is not located in the toilet seat, such as that disposed within another component of the toilet (e.g., a base of the toilet) or disposed in an external device (e.g., compute device 270). In some embodiments, the toilet seat 901 can be configured to transmit sensor data collected by the sensor(s) 910 included in the toilet seat 901 to a processor (e.g., processor 120, 220, 272), such that the processor can process and/or analyze the sensor data, as described above with reference to FIGS. 2A-2B. In some embodiments, the sensing device depicted in FIGS. 9A-9B can be installed on an existing toilet, e.g., by retrofitting an existing toilet.

FIGS. 9A shows the toilet seat 901 includes four sensors and/or electrodes 960 which can be configured to function as impedance sensor(s). The four impedance sensors 960 can be coupled such that the first, second, third, and fourth impedance sensor 960 form and/or constitute a kelvin-like drive circuit and/or a four-electrode configuration. FIG. 9A shows a first and a second impedance sensor 960 can be disposed on a first side of the toilet seat 901. More specifically, the first impedance sensor can be disposed on a rear portion of the first side of the toilet (e.g., see impedance sensor A), while the second impedance sensor is disposed on a front portion of the first side of the toilet (e.g., see impedance B). In use, the first and second impedance sensors are placed in direct contact with a buttock (or any adjacent skin surface including for example, a thigh) of a user when the user is seated on the toilet. FIG. 9A also shows a third and a fourth impedance sensor 960 can be disposed on a second side of the toilet seat opposite to the first side toilet. The third impedance sensor can be disposed on a rear portion of the second side of the toilet (e.g., see impedance sensor C), while the fourth impedance sensor is disposed on a front portion of the second side of the toilet (e.g., see impedance D). In use, the third and fourth impedance sensors are placed in direct contact with the other buttock (or an adjacent skin surface including for example, a thigh) of the user when the user is seated on the toilet. In some implementations the first and third impedance sensors (e.g., the rear impedance sensors A and C) can be configured to act and/or operate as excitation electrodes, which deliver an excitation signal by applying an excitation current trough the first and the third impedance sensors when the user is seated on the toilet. In such implementations, the second and the fourth impedance sensors (e.g., the front impedance sensors B and D) can be configured to act and/or operate as sensing electrodes, which sense and/or measure a response signal in response to the excitation signal (e.g., measure a voltage difference, loss, and/or drop across the first and the third impedance sensor caused by the excitation current). Alternatively, in some implementations the first and third impedance sensors (e.g., the rear impedance sensors A and C) can be configured to act and/or operate as the sensing electrodes, while the second and fourth impedance sensors (e.g., the front impedance sensors B and D) care configured to act and/or operate as the excitation electrodes. The kelvin-like drive circuit configuration described above can facilitate eliminating resistive voltage loses and/or drops caused by the current source, resulting in more accurate impedance measurement. The first, second, third, and fourth impedance sensors described above can be operatively coupled to a processor (e.g., the processor 120, 220, and/or 272) that can receive signals from the first, second, third, and fourth impedance sensors to determine a buttock-to buttock bioimpedance of the user.

Additionally or alternatively, in some embodiments, the first and the second impedance sensors 960 (e.g., impedance sensor A and B) shown in FIG. 9A can disposed such that tissue of a thigh of the user seated on the toilet can be placed in contact with the first and the second impedance sensors. That is to say, a first portion of a thigh of the user can be placed in direct contact with the first impedance sensor (impedance sensor A) while a second portion of that thigh of the user can be placed in direct contact with the second impedance sensor (impedance sensor B). The first and second impedance sensors can be coupled such that the first and second impedance sensors can form a closed-loop circuit. The current and/or voltage associated with the closed-loop circuit can be measured (e.g., the voltage between the first and second impedance sensors can be measured), processed (e.g., amplified, filtered, digitized) and send to a processor (e.g., processor 120, 220, and/or 272). As described above, the processor can be configured to determine a bioimpedance based on the measured current and/or voltage. When a user places one of the user's thigh in contact with the first and second impedance sensor disposed on the toilet seat such that a first portion of the thigh of the user placed in contact with the first impedance sensor (impedance sensor A) and a second portion of that thigh of the user in contact with the second impedance sensor (impedance sensor B), the first impedance sensor can send a signal (e.g., a current) to the second impedance sensor, and a resulting voltage can be measured between the first and the second impedance sensor to determine a bioimpedance of the thigh of the user. Similarly, the third and fourth impedance sensors (e.g., impedance sensor C and D) can be disposed such that tissue of a thigh of the user seated on the toilet can be placed in contact with the third and the fourth impedance sensors, and a bioimpedance of the thigh of the user can be measured. In some embodiments, the impedance sensors 960 shown in FIG. 9A can be configured to determine a buttock-to-buttock bioimpedance of the user as well as a thigh impedance of the user.

FIG. 9B shows the toilet seat 901 include four sensors and/or electrodes 960 which can be configured to function as impedance sensor(s). The four impedance sensors 960 can be coupled such that the first, second, third, and fourth impedance sensor 960 form and/or constitute a kelvin-like drive circuit and/or a four-electrode configuration. FIG. 9B shows a first and a second impedance sensor 960 can be disposed on a first side of the toilet seat 901 side by side. That is to say, the first impedance sensor can be disposed on an outer region close to the exterior edge of the first side of the toilet seat 901 (e.g., see impedance sensor A), while the second impedance sensor can be disposed on an interior region close to the opening 912 (e.g., see impedance sensor B). In use, the first and second impedance sensors are placed in direct contact with a buttock (or any adjacent skin surface, including for example, a thigh) of a user when the user is seated on the toilet. FIG. 9B also shows a third and a fourth impedance sensor 960 can be disposed on a second side of the toilet seat opposite to the first side toilet side by side. The third impedance sensor can be disposed on an interior region close to the opening 912 (e.g., see impedance sensor C), while the fourth impedance sensor can be disposed on an outer region close to the exterior edge of the second side of the toilet seat 901 (e.g., see impedance sensor D). In use, the third and fourth impedance sensors are placed in direct contact with the other buttock (or an adjacent skin surface, including, for example a thigh) of the user when the user is seated on the toilet.

In some implementations the first and fourth impedance sensors (e.g., the outer region impedance sensors A and D in FIG. 9B) can be configured to act and/or operate as excitation electrodes, while the second and third impedance sensors (e.g., the interior region impedance sensors B and C in FIG. 9B) are configured to act and/or operate as sensing electrodes. Alternatively, in some implementations the location of the excitation and sensing electrodes can be reversed. That is to say, the first and fourth impedance sensors (e.g., the outer region impedance sensors A and D in FIG. 9B) can be configured to act and/or operate as sensing electrodes, while the second and third impedance sensors (e.g., the interior region impedance sensors B and C in FIG. 9B) are configured to act and/or operate as excitation electrodes. In yet other implementations, the first and third impedance sensors (e.g., impedance sensors A and C in FIG. 9B) can be configured to act and/or operate as the excitation electrodes, while the second and fourth impedance sensors (e.g., impedance sensors B and D) are configured to act and/or operate as the sensing electrodes. Additionally, in some implementations the location of the excitation and sensing electrodes can be reversed. For example, the first and third impedance sensors (e.g., impedance sensors A and C in FIG. 9B) can be configured to act and/or operate as the sensing electrodes, while the second and fourth impedance sensors (e.g., impedance sensors B and D) are configured to act and/or operate as the excitation electrodes. The kelvin-like drive circuit configuration(s) described above can facilitate eliminating resistive voltage loses and/or drops caused by the current source, resulting in more accurate impedance measurement. The first, second, third, and fourth impedance sensors described above can be operatively coupled to a processor (e.g., the processor 120, 220, and/or 272) that can receive signals from the first, second, third, and fourth impedance sensors to determine a buttock-to buttock bioimpedance of the user.

Additionally or alternatively, in some embodiments, the first and the second impedance sensors 960 (e.g., impedance sensor A and B) shown in FIG. 9B can disposed such that tissue of a thigh of the user seated on the toilet can be placed in contact with the first and the second impedance sensors. That is to say, a first portion of a thigh of the user can be placed in direct contact with the first impedance sensor (impedance sensor A) while a second portion of that thigh of the user can be placed in direct contact with the second impedance sensor (impedance sensor B). The first and second impedance sensors can be coupled such that the first and second impedance sensors can form a closed-loop circuit. The current and/or voltage associated with the closed-loop circuit can be measured (e.g., the voltage between the first and second impedance sensors can be measured), processed (e.g., amplified, filtered, digitized) and send to a processor (e.g., processor 120, 220, and/or 272). As described above, the processor can be configured to determine a bioimpedance based on the measured current and/or voltage. When a user places one of the user's thigh in contact with the first and second impedance sensor disposed on the toilet seat such that a first portion of the thigh of the user placed in contact with the first impedance sensor (impedance sensor A) and a second portion of that thigh of the user in contact with the second impedance sensor (impedance sensor B), the first impedance sensor can send a signal (e.g., a current) to the second impedance sensor, and a resulting voltage can be measured between the first and the second impedance sensor to determine a bioimpedance of the thigh of the user. Similarly, the third and fourth impedance sensors (e.g., impedance sensor C and D) can be disposed such that tissue of a thigh of the user seated on the toilet can be placed in contact with the third and the fourth impedance sensors, and a bioimpedance of the thigh of the user can be measured. In some embodiments, the impedance sensors 960 shown in FIG. 9B can be configured to determine a buttock-to-buttock bioimpedance of the user as well as a thigh impedance of the user.

In some embodiments the impedance sensors and/or electrodes described above with reference to FIGS. 9A-9B can be configured to generate an excitation signal and measure a response signal to determine a bioimpedance of the user at a predetermined frequency. In some embodiments the impedance electrodes and/or sensors described above can be configured to determine a bioimpedance of the user at multiple frequencies. For example, in some embodiments the impedance electrodes and/or sensors can be configured to determine a bioimpedance of a user at multiple frequencies between 40 Hz and 10 MHz. In some embodiments, the impedance electrodes and/or sensors can be configured to determine bioimpedance data of the user sequentially (e.g., one frequency at a time producing a frequency sweep). Alternatively, the impedance electrodes and/or sensors can be configured to determine bioimpedance data of the user at different frequencies simultaneously. In some embodiments the impedance electrodes and/or sensors can be configured to determine bioimpedance data of the user continuously while the user is seated on the toilet. In other embodiments the impedance electrodes and/or sensors can be configured to determine bioimpedance data of the user throughout random periods of time while the user is seated on the toilet.

In some embodiments, two of the sensors 960 shown in FIG. 9A or FIG. 9B can be configured to function as ECG sensors. In such embodiments, the two sensors 960 (e.g., sensor A and C, sensor B and D, sensor A and D, or sensor B and C) can be configured to measure signals representative of the electrical activity of the heart of a user and/or subject originating from the depolarization of the conductive pathway of the heart and the cardiac muscle tissues during each cardiac cycle, as described above with reference to the ECG sensors 140 and 340 in FIGS. 1 and 3A-3C.

In some embodiments, three of the sensors 960 shown in FIG. 9A or FIG. 9B can be configured to function as ECG sensors. In such embodiments, the three sensors and/or electrodes 960 can be configured as a 3-electrode system. The 3-electrode system can include a first and a second electrode (e.g., electrode A and C) which can be and/or function as single lead signal ECG electrodes; and a third electrode (electrode B) which can be a driven right leg electrode that functions as a reference point for the ECG signal(s). The first, second, and third electrodes can be operatively coupled to a processor (e.g., the processor 120) that can receive signals from the first, second, and third electrode to determine ECG data and/or a bioimpedance of a user when the user is seated on the toilet.

Systems and devices described herein, including those described in FIGS. 1-3, and 9, can actively and/or passively determine an identity of an individual or subject seated on a toilet seat or other lavatory device. In some embodiments, systems and devices described herein may implement an initialization or onboarding process for new individuals to be able to identify those same individuals at later times. In particular, systems and devices described herein may obtain sensor data of a user (e.g., during a training period or under different conditions) and associate that sensor data with a subject. Such associated data can then be stored and used as reference data at future times to identify a user. In some embodiments, such associated data can represent a biological profile or bio-identification of a user, which can be referenced at future times to identify a user. FIGS. 4A-4B provide an example of an initiation or onboarding process 400, according to an embodiment. The process 400 can be implemented by a processor, including, for example, a processor onboard a sensing system or device (e.g., a processor similar to processor 120 and/or 220 described above with reference to FIGS. 1 and 2) or a processor of a remote compute device to which a sensing system is coupled (e.g., processor 272 of a compute device 270 described above with reference to FIGS. 2A-2B).

As depicted in FIG. 4A, the onboarding process 400 can include beginning with optionally prompting a user to provide identification information at step 401. In some embodiments, the processor can communicate with the user via a communications interface (e.g., communications interface 126 or 226) of the sensing system. For example, the communications interface can include an output device that can present (e.g., via audio or video) the prompt to the user. Alternatively, the communications interface can communicate the prompt to an external device (e.g., a user device 280, a compute device 270, and/or a third-party device 290) to have the external device present the prompt to the user. The processor can prompt the user to provide identification information such as, for example, a name, a birthdate, a username or user identifier, or other information that can be used to identify the user. In instances where the user is a new user, the user can create a user account and associate their identity to a username or user identifier of the account.

At step 402, the onboarding process 400 can optionally include prompting a caretaker of the user to provide identification information. In some embodiments, the processor can communicate with a caretaker, a healthcare worker, and/or a third party associated with the user via the communications interface (e.g., communications interface 126 or 226) of the sensing system, to prompt and/or request the caretaker, healthcare worker, or third party to provide the identification information of the user. In some instances, the caretaker can remotely (e.g., at a location different from the location in which the user and the sensing system is located) enter all the identification information on behalf of the user of the sensing system 200. Furthermore, in some embodiments, the caretaker, healthcare worker, or third party can populate the information of the user prior to the user being introduced to the sensing system 200.

The method 400 includes conducting one or more recordings of the user or subject seated on a toilet or other lavatory device. For example, a sensing system (e.g., sensing system 100, 200, or 300) can measure and record sensor data when a user is seated on a toilet, at 403. As described above, the sensing system can include sensors that are configured to measure and record data of the user. The processor (e.g., an onboard or remote processor) can receive the measured data from the sensing system. In some embodiments, the processor can be configured to analyze the sensor data and determine physical and/or physiological information of the user, including, for example, a seated weight, a total weight, an ECG feature, a PPG feature, a bioimpedance, etc.

In some embodiments, instead of prompting a user for identification information, at step 401 or 402, the processor can also automatically compare the sensor data measured of the user at step 404 to previously collected and stored sensor data of known users. If, based on the comparison, the processor determines that the sensor data of the user is similar to that of a known user (e.g., via statistical and/or machine learning algorithms), at 405, the processor can determine that the user is the known user. The processor can then retrieve the user identifier associated with the known user. Further details of how a user can be identified based on sensor data are described with reference to FIG. 5 below.

At 406, the processor can then associate the sensor data (e.g., raw or filtered) of the user with the user identifier associated with that user. As described above, the user identifier may have been entered by the user or created by the user or a caretaker of the user, at steps 401 or 402, or the user identifier can be retrieved by the processor after the processor identifies the user as a known user, at 405. In some embodiments, the processor may determine or extract information from the sensor data of the user (e.g., information of one or more physiological characteristics, parameters, or conditions of the user), and such determined or extracted information may be associated with the user, in lieu of or in addition to raw or filtered sensor data.

In some embodiments, where the processor does not prompt the user to provide or create a user identifier, and the processor does not determine that the sensor data is similar to previously collected sensor data of known users, then the processor can create a new user identifier for the user and associate the sensor data of the user with the new user identifier, at 407.

Optionally, the process 400 can include collecting sensor data of the user under different conditions. For example, as shown in FIG. 4B, the processor can be configured to collect senor data of the user when the user is seated at different postures on the toilet seat at step 410. The user can be instructed to adjust his posture and to remain in each posture for a predetermined period of time (e.g., seconds, a minute, or minutes) so that the sensing system can measure data of the user in each posture, at 411. In some embodiments, the processor can communicate with the user via the communication interface of the sensing system, to instruct the user to hold each of a plurality of postures for a predetermined period of time. In some embodiments, the processor can transmit a message to an external device (e.g., a user device, compute device, and/or third-party device), instructing the user to hold each of a plurality of postures for a predetermined period of time. The external device can be configured to receive the message from the sensing system and present it to the user e.g., via a display, audio device, or other output component of the external device. Examples of postures that the user may be instructed to be in include: sitting on the toilet with upper body leaning forward such that the weight of the user is closer to the front side of the toilet, sitting on the toilet with upper body leaning backward, sitting on the toilet with upper body leaning to a right or left side, sitting on the toilet with upper body being erect or as straight as possible, etc. In other instances, the sensing system can present a message to the user to instruct the user to seat on the toilet with the user's upper body leaning forward.

At 412, the sensing system can record sensor data of the user at the different postures (e.g., the postures instructed to the user at 411). At 413, the processor can associate the sensor data recorded at the different postures with the user, e.g., by associating it with the user identifier of the user.

After associating the sensor data with the user identifier at 407 or 413, the processor can be configured to store the sensor data in a memory (e.g., an onboard memory or a remote memory or database coupled to the processor). This stored sensor data can then be used as reference data (or a biological profile) to compare with newly acquired sensor data to facilitate identification of future unknown users, as further described herein.

Optionally, the process 400 can include collecting sensor data associated to the patterns of the user when sitting on the toilet. These patterns, which can also be referred to as landing patterns correspond to the different positions and/or orientations that the user may assume when siting on the toilet. In some embodiments, the landing patterns of a user can be recorded by the processor and used to determine the identity of the user. As shown in FIG. 4B, the processor can be configured to collect sensor data of the landing patterns of the user when the user sits on the toilet seat at step 420. The user can be instructed to stand up and then sit on the toilet approaching the toilet according to different orientations, positions and/or postures. For example, the user can be instructed to approach the toilet from the front of the toilet and sit on the toilet while assuming a leaning forward orientation. Alternatively, the user may be instructed to approach the toilet from different orientations (e.g., at an angle, or sideways) while assuming different orientation. The user can be instructed to repeat instructing the user to stand up and sit on the toilet multiple times so that the sensing system can measure sufficient data of the user in each landing, at step 421.

At 422, the sensing system can record sensor data of the user sitting pattern (e.g., the stand up and sit repetitions instructed to the user at 421). At 423, the processor can associate the sensor data of the user sitting pattern, e.g., by associating it with the user identifier of the user.

After associating the sensor data with the user identifier at 407 or 413, the processor can be configured to store the sensor data in a memory (e.g., an onboard memory or a remote memory or database coupled to the processor). This stored sensor data can then be used as reference data (or a biological profile) to compare with newly acquired sensor data to facilitate identification of future unknown users, as further described herein.

While the onboarding process depicted in FIGS. 4A-4B shows that a single measurement of a user can be collected, or multiple measurements collected at different postures, it can be appreciated that any number of measurements of a user can be collected during the onboarding process. For example, a user may be instructed to sit on the toilet for an extended period of time so that n measurements of the data can be taken during that period of time. In some embodiments, the onboarding process may last over a few sitting sessions. For example, a user may be asked to identify himself during multiple sitting sessions on a toilet (such as, for example, the first 3-10 sessions), such that the processor can associate the data measured during those multiple sessions with the user. Having multiple sets of data be collected of a user can increase the robustness of the processor in identifying the user at later times.

FIG. 5 depicts a flow chart of a process 500 for identifying a user based on sensor data measured of the user, according to embodiments. The process 500 can be implemented by a processor, including, for example, a processor onboard a sensing system or device (e.g., a processor similar to processor 120 and/or 220 described above with reference to FIGS. 1 and 2) or a processor of a remote compute device to which a sensing system is coupled (e.g., processor 272 described above with reference to FIGS. 2A-2B).

As depicted in FIG. 5, several different types of sensor data, e.g., force data, ECG data, PPG data, and/or impedance data, can be recorded by a sensing system (e.g., sensing system 100, 200, or 300). In some embodiments, only a single type of sensor data may be recorded, while in other embodiments, two or more types of sensor data may be recorded.

At 501, the sensing system can record force data of a user seated on a toilet. In particular, the sensing system can include force sensors that are disposed around a toilet ring, and those force sensors can capture force data of the user when the user is seated on the toilet ring. The force data can be provided to the processor for further processing and/or analysis. In some embodiments, the processor may process the raw force data to remove artifacts and/or noise. In some embodiments, the processor may process the raw data to provide BCG data of the user. In some embodiments, the processor can optionally determine one or more BCG related features, and/or a posture or dynamic weight of the user using the force data, at 502. The BCG related features can include, for example, an ejection wave (e.g., I and/or J ejection waves), as well as one or more pre-systolic peaks, systolic peaks, and/or diastolic peaks. In particular, the processor can determine a distribution of the weight of the user, where the distribution indicates how the user's weight is concentrated around the toilet seat. Such weight distribution can be indicative of a posture of the user (e.g., user leaning forward, user leaning back, user leaning to the left, user leaning to the right, or user sitting upright). In some embodiments, the sensing system (or a separate sensing system) can include one or more force sensors that are disposed in a scale assembly. The force sensors in the scale assembly together with the force sensors in a toilet ring can provide more comprehensive weight distribution data of the user. In such embodiments, the processor can determine a posture and/or weight distribution of the user based on the force data captured along the toilet ring and by the scale assembly. For example, when more of the user's weight is concentrated on the toilet ring, the processor may determine that the user is leaning or sitting back on the toilet. Alternatively, when more of the user's weight is concentrated on the scale assembly, the processor may determine that the user is leaning forward (e.g., in a reading position). In some embodiments, the processor can optionally determine a seated weight of the user and/or a BCG waveform of the user, at 503. For example, the processor can segment the raw or filtered BCG signal into fixed or time-varying windows, and then average the segmented BCG signal across the windows to determine an average BCG waveform of the user. The processor, via the force data collected by the sensors along the toilet ring, can determine a partial, seated weight of the user. At 504, the processor can compare the force data of the user and/or one or more determined characteristics to stored reference data of known users. For example, the processor can determine a correlation between the force data of the user to stored force data of a plurality of known users using one or more statistical models (e.g., log likelihood function, joint Gaussian distribution). In some embodiments, the processor can process the force data of the user using a trained classification model that classifies the user as one of the known users or an unknown user. The classification model may have been previously trained using force data and/or other data of the known users. The processor can then evaluate whether the correlation or classification is indicative of the user being one of the known users (e.g., based on strength of correlation, degree of confidence, etc.), at 550.

At 520, the sensing system can record ECG data of the user seated on the toilet. In particular, the sensing system can include ECG sensors that are disposed on a top face of a toilet ring. The ECG sensors can come into contact with the buttocks or legs of a user when the user is seated on the toilet, and such contact can enable the sensors to capture ECG data of the user. Alternatively, in some embodiments the sensing system can include ECG sensors disposed on one or more handles attached to the toilet seat, a wall disposed near the toilet, and/or at any suitable location such that a user can hold the handles while sitting on the toilet. The user can hold and/or grab the handles such that the ECG sensor can come into contact with one or the two hands of the user, enabling the sensor to capture ECG data of the user. The ECG data can be provided to the processor for further processing and/or analysis. In some embodiments, the processor may process the raw ECG data to remove artifacts and/or noise. In some embodiments, the processor can optionally determine one or more ECG related features, including, for example, a R-peak amplitude, a R-R interval, a power of the ECG, a heart rate, etc., or averages thereof, at 521. In some embodiments, the processor can optionally determine an ECG waveform of the user, at 522. For example, the processor can segment the raw or filtered ECG signal into fixed or time-varying windows, and then average or perform other statistical measures on (e.g., higher order statistics, peak amplitudes, slope, area under curve, etc.) the segmented ECG signal across the windows to determine an average ECG waveform or other ECG data of the user. In some embodiments, the process can segment one or more regions of an ensemble average or an ensemble median ECG based on time windows and/or features (e.g., QRS complex), and then average (or perform other statistical measures on) the ECG signal within those regions. At 524, the processor can compare the ECG data of the user and/or one or more determined characteristics to stored reference data of known users. For example, the processor can determine a correlation between the ECG data of the user to stored ECG data of a plurality of known users using one or more statistical models. In some embodiments, the processor can process the ECG data of the user using a trained classification model that classifies the user as one of the known users or an unknown user. The classification model may have been previously trained using ECG data and/or other data of the known users. The processor can then evaluate whether the correlation or classification is indicative of the user being one of the known users (e.g., based on strength of correlation, degree of confidence, etc.), at 550.

At 530, the sensing system can record PPG data of the user seated on the toilet. In particular, the sensing system can include PPG sensors that are disposed in a toilet ring. The PPG sensors can detect light reflected by the buttocks or legs of a user when the user is seated on the toilet to capture PPG waveform data. In some embodiments, the sensors can detect light reflected by the buttocks which corresponds to one or more specific wavelengths. For example, in some embodiments the sensors can detect reflected red light, infrared light, and/or green light. The PPG data can be provided to the processor for further processing and/or analysis. In some embodiments, the processor may process the raw PPG data to remove artifacts and/or noise. In some embodiments, the processor can optionally determine one or more PPG related features, including, for example, a DC amplitude, a systolic peak (SP) amplitude, a SP-SP interval, a heart rate, etc., or averages thereof, at 531. In some embodiments, the processor can determine the PPG related features from reflected light of multiple wavelengths (e.g., red light, infrared light, and/or green light), and combine those PPG related features to generate new features that facilitate identifying a user. For example, in some embodiments the processor can determine a DC amplitude from reflected red light and a DC amplitude from reflected infrared light. The processor can then determine a DC amplitude ratio (e.g., DC red light/DC infrared light) which can be used to differentiate a user. In some embodiments, the processor can optionally determine a PPG waveform of the user, at 532. For example, the processor can segment the raw or filtered PPG signal into fixed or time-varying windows, and then average or perform other statistical measures on (e.g., higher order statistics, peak amplitudes, slope, area under curve, etc.) the segmented PPG signal across the windows to determine an average PPG waveform or other PPG data of the user. In some embodiments, the process can segment one or more regions of an ensemble average PPG based on time windows and/or features (e.g., systolic peak, diastolic peak, etc.), and then average (or perform other statistical measures on) the PPG signal within those regions. At 534, the processor can compare the PPG data of the user and/or one or more determined characteristics to stored reference data of known users. For example, the processor can determine a correlation between the PPG data of the user to stored PPG data of a plurality of known users using one or more statistical models. In some embodiments, the processor can process the PPG data of the user using a trained classification model that classifies the user as one of the known users or an unknown user. The classification model may have been previously trained using PPG data and/or other data of the known users. The processor can then evaluate whether the correlation or classification is indicative of the user being one of the known users (e.g., based on strength of correlation, degree of confidence, etc.), at 550.

At 540, the sensing system can record impedance data of the user seated on the toilet. In particular, the sensing system can include electrodes or conductive elements that can measure an impedance associated with the user when the user's skin is in contact with the conductive elements. The conductive elements can form a closed circuit with a portion of the user's anatomy (e.g., thigh-to-thigh, foot-to-foot) to measure a bioimpedance of the user. The bioimpedance data can be provided to the processor for further processing and/or analysis. At 544, the processor can compare the bioimpedance data of the user and/or one or more determined characteristics to stored reference data of known users. For example, the processor can determine a correlation between the bioimpedance data of the user to stored bioimpedance data of a plurality of known users using one or more statistical models. The bioimpedance data can include amplitude and frequency information, which can be used to identify the user. For example, in some embodiments the processor can compare the average impedance of a user at a predetermined frequency, with stored average impedance of known users at the predetermined frequency to identify the user. In some embodiments, the processor can process the bioimpedance data of the user using a trained classification model that classifies the user as one of the known users or an unknown user. The classification model may have been previously trained using bioimpedance data and/or other data of the known users. The processor can then evaluate whether the correlation or classification is indicative of the user being one of the known users (e.g., based on strength of correlation, degree of confidence, etc.), at 550.

In response to determining whether the correlations meet one or more predetermined criteria, at 550, the processor can identify the user. For example, when the correlation with the reference data of a known user is sufficiently high (e.g., above a predefined threshold), or when multiple types of data correlate sufficiently high (e.g., above one or more predefined thresholds), then the processor can identify the user as that known user, at 551. When the correlations are low or fail to satisfy the predetermined criteria, then the processor may indicate to the user that it has not been able to identify the user and/or prompt the user to provide identification information (e.g., similar to that described with respect to step 401 or 402 of FIG. 4A), at 552.

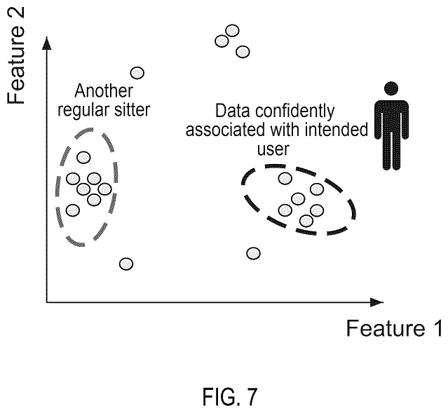

For illustrative purposes, FIG. 7 depicts an example of data points of different users plotted on a scatter plot, according to embodiments. Each data point can represent or be indicative of one or more physiological characteristics, conditions, and/or other information of a user. For example, the scatter plot can represent a way to visualize the physiological characteristics, conditions, and/or other information of users, where multiple features associated with the physiological characteristics, conditions, and/or other information of the users assigned different axes and data of each user is plotted accordingly on those axes. Each data point may be associated with a different measurement taken of the user, e.g., using sensing systems or devices as described herein. For example, each data point may be associated with a measurement taken of a user at different times he has used a toilet or other lavatory device (e.g., during differing sitting sessions at a toilet). Alternatively or additionally, for users that may have been instructed to undergo an initiation process as described above in FIGS. 4A-4B, one or more data points of that user may be associated with different phases of the initiation process (e.g., when the user is instructed to sit at different postures, or when the user is instructed to take n measurements, as described above). As shown in FIG. 7, a first user (e.g., an intended user) may have a plurality of data points that can be grouped together, e.g., based on a cluster analysis. Similarly, a second user (e.g., another regular sitter) may have a plurality of data points that can be grouped together, e.g., based on cluster analysis. Given the grouping of the data points for both users, systems and devices described herein can be configured to determine whether a new data point of an unknown user is sufficiently similar to (e.g., correlated with) the data points of either user to identify the unknown user as the first user, the second user, or a new user. It can be appreciated that while the data of two users is depicted in FIG. 7, the data of any number of users can be stored by systems and devices described herein and referenced, when new data is measured of a user, to identify that user.

Referring back to FIG. 5, while sub-processes 501-504, 520-524, 530-534, and 540-544 are depicted as separate sub-processes, it can be appreciated that one or more of these processes can be performed together, e.g., sequentially and/or concurrently. Moreover, in some embodiments, one or more of force data (including BCG data), ECG data, PPG data, bioimpedance data, and/or characteristics determined therefrom can be combined or evaluated together and used to determining an identity of a user (e.g., by comparing to or correlating with reference data). For example, in some embodiments, a seated weight and the distribution of weight or posture may be evaluated together to determine an identity of a user. In some embodiments, a seated weight and bioimpedance of a user may be evaluated together to determine an identity of a user. In some embodiments, a seated weight and an ECG of a user may be evaluated together to determine an identity of a user.

In some embodiments, the sensor data that is measured of a user during a live session can be associated with the identified user (e.g., associated with a user identifier of the user), and stored to be used as future reference data of that user. For many users, their physiological data may change over time, e.g., based on changing circumstances and/or health. As such, unobtrusive health monitoring systems that a user would commonly interface with, e.g., such as a toilet or other lavatory device, can be well suited to track the changes in physiological data of such users.

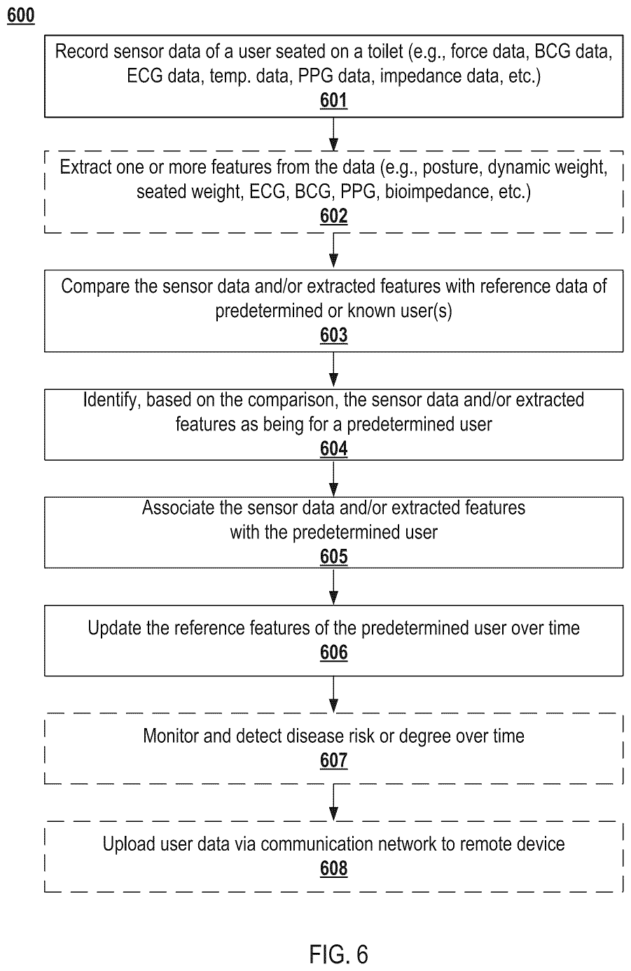

FIG. 6 depicts a processor 600 of identifying a user, where a processor can track the data of a user to passively adapt a biological profile or bio-identification of the user over time, according to embodiments. The process 600 can be implemented by a processor, including, for example, a processor onboard a sensing system or device (e.g., a processor similar to processor 120 and/or 220 described above with reference to FIGS. 1 and 2A-2B) or a processor of a remote compute device to which a sensing system is coupled (e.g., processor 272 described above with reference to FIG. 2). The process 600 can include one or more parts that are similar to processes 400 and/or 500, described above. As such, certain details of these parts are not provided in detail herein again.

At 601, a sensing system (e.g., sensing system 100, 200, or 300) can record sensor data (e.g., force data, BCG data, ECG data, temp. data, PPG data, impedance data, etc.) when a user is seated on a toilet, e.g., similar to that described with respect to 401 of process 400. The sensing system can provide this data to the processor. At 602, the processor optionally can extract one or more features from the sensor data (e.g., posture, dynamic weight, seated weight, ECG, BCG, PPG, bioimpedance, etc.). At 603, the processor can automatically compare the sensor data of the user and/or one or more extracted features from the sensor data to previously measured and stored sensor data and/or extracted features, e.g., similar to that described with respect to 403 and 404 of process 400 and/or that described with respect to 504, 524, 534, and 544 of process 500. As described above, in some embodiments, the processor can perform the comparison by evaluating a correlation of the sensor data or extracted features of the user with reference data of one or more known users. Alternatively or additionally, in some embodiments, the processor can perform the comparison by processing the data using one or more machine learning algorithms (e.g., a classification algorithm).

At 604, the processor can identify, based on the output of the comparison, whether the sensor data and/or extracted features are likely that of a known user, e.g., similar to that described with respect to 404 of process 400 and/or that described with respect to 550 of process 500. The processor can then associate the sensor data and/or extracted features with the known user, at 605, e.g., similar to that described with respect to 404 of processor 405.

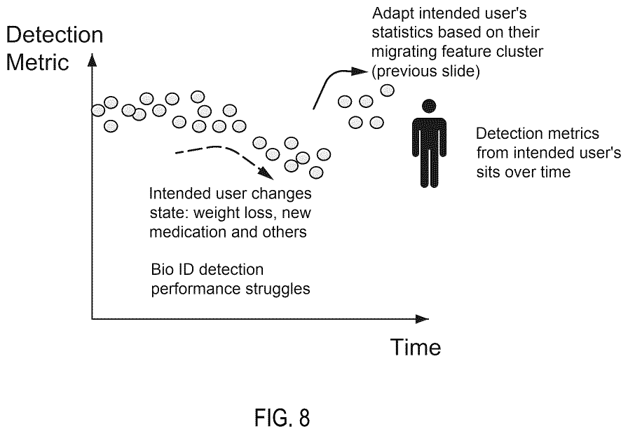

By constantly or repeatedly associating new sensor data of a user with that user (e.g., by associating the new sensor data with the user's identifier), the processor can be configured to adapt a biological profile or bio-identification of the user over time. At 606, the processor can be configured to update the reference data of the user over time. For example, as illustratively shown in FIG. 8, the data points associated with a user may migrate over time. FIG. 8, similar to FIG. 7, depicts an example of data points of users plotted on a scatter plot. Each data point can represent or be indicative of one or more physiological characteristics, conditions, and/or other information of a user. Over time (e.g., over a plurality of sitting sessions), a user may change state, e.g., due to weight low, new medication, new environment, etc. These changes are reflected in the data points of that user.

Referring back to FIG. 6, the processor can be configured to track these physiological changes of a user over time and to update one or more reference features of the user, at 606. In some embodiments, the processor can be configured to monitor and detect disease risk of degree over time, at 607. For example, the processor can be configured to compare the user's sensor data (or information extracted therefrom) to predetermined metrics indicative of various health conditions. As a user's data migrates towards a predetermined threshold or other metric that is indicative of a health condition, the processor can be configured to alert a user of such migration. In some embodiments, the processor can be configured to communicate with one or more other compute devices (e.g., a user device or a third-party device) to inform a user or third-party clinicians or caregivers of the user's condition, at 608.

While systems, devices, and methods are described herein with respect to a toilet seat or lavatory, it can be appreciated that systems, devices, and methods described herein can be implemented in other types of devices. For example, systems, devices, and methods can perform user identification with other types of sensing systems, including, wearable devices (e.g., armbands, wristbands, headbands, glasses, etc.), scales, exercise equipment, etc.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A sensing system, comprising:
a set of sensors disposed on a seat coupled to a waste receptable and including a surface on which a user can be seated, the set of sensors configured to measure sensor data present on the seat when the user is seated on the seat; and
a processor operatively coupled to the set of sensors, the processor configured to:
receive signals indicative of the measured sensor data;
determine, in response to receiving the signals, one or more physiological characteristics about the user based on the measured sensor data;
correlate the determined one or more physiological characteristics with reference data of previous users stored in a memory; and
identify the user based on the correlation as one of the previous users.

2. The sensing system of claim 1, wherein the set of sensors include force sensors configured to collectively measure forces present on the seat when the user is seated on the seat.

3. The sensing system of claim 2, wherein the set of sensors further include a scale device configured to measure forces present on the scale device when the user is seated on the seat and one or more feet of the user is placed on the scale device.

4. The sensing system of claim 2, wherein the one or more physiological characteristics include at least one of a seated weight, a ballistocardiogram (BCG), or a posture of the user.

5. The sensing system of claim 1, wherein the set of sensors further includes a temperature sensor configured to measure a core body temperature of the user.

6. The sensing system of claim 1, wherein the set of sensors further includes an electrocardiogram (ECG) sensor.

7. The sensing system of claim 6, wherein the ECG sensor includes:
a first lead electrode configured to be in contact with a thigh of the user;
a second lead electrode configured to be in contact with a first portion of another thigh of the user; and
a driven-right-leg (DRL) electrode configured to be in contact with a second portion of the another thigh of the user.

8. The sensing system of claim 1, wherein the set of sensors further includes a photoplethysmography (PPG) sensor.

9. The sensing system of claim 1, wherein the set of sensors further includes one or more impedance sensors disposed on the seat.

10. The sensing system of claim 9, wherein the one or more impedance sensors include four impedance sensors, the four impedance sensors forming drive circuit configured to determine a bioimpedance of the user when the user is seated on the seat.

11. A method, comprising:
recording, with a sensing system including a set of sensors disposed on a seat of a toilet, sensor data of a user seated on the toilet;
extracting, with a processor operably coupled to the set of sensors, one or more features of the recorded sensor data;
comparing, with the processor, the sensor data or the one or more features with reference data of a plurality of known users; and
determining, with the processor and based on the comparing, an identity of the user seated on the toilet.

12. The method of claim 11, wherein determining an identity of the user includes:
determining a correlation between the sensor data or the one or more features of the recorded sensor data with the reference data of the plurality of known users; and
evaluating, based on a statistical model, if the correlation is indicative of the user being one of the plurality of known users.

13. The method of claim 11, wherein the set of sensors include force sensors and the sensor data includes forces present on the seat measured by the force sensors when the user is seated on the seat.

14. The method of claim 13, wherein the set of sensors further include a scale device and the sensor data includes forces present on the scale device measured by the scale device when the user is seated on the seat and one or more feet of the user is placed on the scale device.

15. The method of claim 13, wherein the one or more features of the recorded sensor data include at least one of a seated weight, a ballistocardiogram (BCG), or a posture of the user.

16. The method of claim 11, wherein the set of sensors further includes a temperature sensor and the sensor data include a core body temperature of the user measured by the temperature sensor.

17. The method of claim 11, wherein the set of sensors further includes an electrocardiogram (ECG) sensor.

18. The method of claim 11, wherein the set of sensors further includes a photoplethysmography (PPG) sensor.

19. The method of claim 11, wherein the set of sensors further includes one or more impedance sensors.

20. The method of claim 19, wherein the one or more impedance sensors include four impedance sensors, the four impedance sensors forming drive circuit configured to determine a bioimpedance of the user when the user is seated on the seat.
